(19) United States
(12) Patent Application Publication
(10) Pub. No.: US 2023/0338975 A1
(43) Pub. Date: Oct. 26, 2023

(54) FLUID DISPENSING APPARATUS

(71) Applicant: 1TOUCH Holdings Inc, Hartford, CT (US)

(72) Inventor: Jim Warner, Chicago, IL (US)

(21) Appl. No.: 18/188,818

(22) Filed: Mar. 23, 2023

Related U.S. Application Data
(63) Continuation-in-part of application No. 17/543,352, filed on Dec. 6, 2021.
Application No. 17/543,352 is a continuation-in-part of application No. 16/507,000, filed on Jul. 9, 2019, now Pat. No. 11,192,705.
(60) Provisional application No. 63/322,824, filed on Mar. 23, 2022.
(60) Provisional application No. 62/695,306, filed on Jul. 9, 2018.

(51) Int. Cl. B05B 11/10; G01F 11/08

(52) U.S. Cl. CPC B05B 11/1028; G01F 11/08

(57) ABSTRACT

An apparatus and method of using a device for dispensing fluid comprising a flexible and collapsible container, a fluid dispenser pump connected to the flexible and collapsible fluid container configured to draw fluid from the flexible and collapsible fluid container into the fluid dispenser pump and to dispense fluid through a fluid delivery channel to an exit port upon actuation of the pump. The dispenser pump having an actuator comprising a dose delivery button, and a sensor located in the apparatus and configured to actuate the dispenser pump upon physical contact of the dose delivery button by a user.

### CROSS-REFERENCE TO RELATED APPLICATIONS

This application claims the benefit of U.S. Provisional Application No. 63/322,824, filed Mar. 23, 2022, the disclosure of which is incorporated herein by reference in its entirety.

### BACKGROUND

The embodiments relate to methods and devices for accurate dispensing, tracking, and reordering of dispensed fluids. Additionally, the embodiments relate generally to fluid dispensers, and more particularly to a fluid dispenser for a portable flexible package having an actuator configured for single-dose dispensing.

The technical problems addressed by the embodiments include: the delivery of information to users of fluids, ensuring that users of fluids use an accurate measurement(s) at the specified time, GPS location of fluid dispensing activity, accurate tracking of the time and dose, enable reminders for use and amount of the dosage to prevent overdose and to track compliance, and prompt for reorder to ensure ongoing compliance for which there is no current solution available, and communication of tracking information to the user, physicians, health care providers, retailers, manufacturers and others who wish to monitor usage, manage compliance, and reorder the fluid. Smart 1 TOUCH will integrate to electronic data collection, smart devices and audible data devices.

Additionally, various types of fluid material and media are employed for different purposes through commerce and industry. For example, there are various products in the personal care, home care, air care, transportation care, and food industries that require some type of dispensing of a fluid material from a source of such material. When this material is sold in commerce, it must be contained and stored in some type of container. When that product is used, it must be dispensed from its storage container to a location for use.

In the prior art, there are many different types of dispenses for delivering fluid material. For example, a flexible container body with a nozzle tip is commonly provided for such a purpose. An application of such use is for the dispensing of ketchup where the container body is squeezed by the user to urge the fluid material out from the nozzle tip and accurately to a desired location. The amount of fluid delivered is determined by how much the user squeezed the container body. However, this yields erratic results where more or less fluid material is delivered on each successive squeeze of the container body. Also, the container must be held upright to avoid leakage because no valves are employed.

In another example of a prior art dispensing device, a flexible container holds a volume of fluid material to be delivered. A single one-way check valve is provided as an exit port from the flexible container. When the flexible body is squeezed, the material is urged out under pressure through the valve.

### SUMMARY

The embodiments address the technical problems described above by either combining a mechanical measured fluid dispenser or sensor technology, such as Bluetooth or RFID, with hardware and software features that detect the time and amount of a fluid dispensing operation, and communicate that information to a user's smart phone app or software which can then optionally, and preferably according to a user-selection, communicate that information to the user's physician, pharmacy, retailers, manufacturers or other interested parties requiring compliance, monitoring and reordering capabilities.

According to various embodiments, the mechanical or sensor based measured fluid dispenser may be of the type disclosed in, for example, U.S. Pat. Nos. 8,123,073, 7,419,322, 8,387,833, 10,259,645, 10,654,059, 10,160,587 and/or 11,008,156, the disclosures of each of which are incorporated herein by reference in their entirety.

According to further embodiments, the sensor or mechanical measured fluid dispenser may include a chip or other sensor configured to detecting a physical change caused by the depression of the dispenser's actuator. For example, the sensor may be a pressure sensor, or a flow rate sensor, a timer, or some combination thereof. The sensor may be any single sensor or combination of sensors that provide information concerning the amount of fluid that is dispensed upon the activation of the dispenser's actuator. Accordingly, the sensor may be located inside or beneath the actuator itself, located somewhere in the fluid channel/pathway, or in dispenser case enclosures.

According to another embodiment, the mechanical measured dispenser may include an adjustable dose mechanism according to which the amount of the dose may be adjusted by the user. According to a further feature of this embodiment, one or more sensors may be provided to determine the adjustment state of the adjustable dose feature. According to one embodiment, the sensor that determines the adjustment state of the adjustable dose mechanism may be separate from and optionally in addition to the sensor(s) that detects and/or measures the amount of a dose when the dose actuator is actuated.

According to a further embodiment, the measured dosing device may be reusable, according to which it may be removed from a first fluid pouch when the first fluid pouch is empty and attached to a second fluid pouch with a fresh supply of fluid.

According to further embodiments, the fluid pouches may include a smart label and/or RFID tag. According to aspects of these embodiments, a user may scan the smart label or RFID tag with a smart phone app which would then cause information concerning the fluid to be displayed to the user. Such information might include how to administer the fluid by using the mechanical measured dispenser or pouch dispenser, how to use the app remind the user to administer the fluid, track compliance, location of where to reorder, automatically reorder, or identify location where fluid can be obtained, how to use the app to track transmit dosage and time information and/or to transmit such information to interested parties, according to the user's preferences and selections.

In another embodiment, the disclosure may be directed to an apparatus and method for dispensing fluid comprising a flexible and collapsible fluid container, a fluid dispenser pump connected to the flexible and collapsible fluid container configured to draw fluid from the flexible and collapsible fluid container into the fluid dispenser pump and to dispense fluid through a fluid delivery channel to an exit port upon actuation of the pump, the dispenser pump having an actuator comprising a dose delivery button, and a sensor located in the apparatus and configured to actuate the dispenser pump upon physical contact of the dose delivery button by a user. The method of using comprises providing the fluid dispensing apparatus, contacting by the user the dose delivery button, signaling the sensor to actuate the fluid dispensing pump, and dispensing fluid from the fluid container through the exit port.

In a further embodiment, the disclosure may be directed to an apparatus and method of using a fluid dispensing device comprising a generally cylindrical base unit having a generally flat and continuous bottom surface, a top surface having a raised circumferential wall, and a center area, a flexible dosing dome configured to sit inside the center area of the base unit to form a pump chamber, the bottom surface of the base unit having a fluid inlet opening to the pump chamber to permit the flow of fluid there-through, the base also defining a fluid delivery channel between the pump chamber and a fluid delivery outlet opening on an outside surface of the base unit, a combination dial and button having the shape of a cylinder with a closed top and open bottom, a bottom portion of the combination dial and button configured to sit inside a channel defined by the base unit top surface and the flexible dome, said button including a Graphical User Interface in communication with a sensor to adjust by a user an amount of a dose of fluid to be dispensed, the sensor located in the device and further configured to actuate the pump chamber upon physical contact of the dose delivery button by the user. The method comprises providing the fluid dispensing device described above, selecting from the Graphical User Interface on the button by the user the adjustment state of the actuator, signaling to the sensor the adjustment state of the actuator to adjust the amount of the dose of fluid dispensed upon actuation, contacting, by the user, the dose delivery button to send a signal to the sensor, and signaling actuation of the pump chamber by the sensor upon contact of the button, wherein the pump chamber dispenses the selected dose of fluid to the outside surface of the base unit.

In yet another embodiment, the disclosure may be directed to an apparatus and method for dispensing and monitoring fluid dispensed comprising a flexible fluid container, a fluid dispenser pump connected to the flexible and collapsible fluid container configured to draw fluid from the flexible and collapsible fluid container into the fluid dispenser pump and to dispense fluid through a fluid delivery channel to an exit port upon actuation of the pump, a sensor located in the apparatus and configured to actuate the dispenser pump upon a first voice command received by the sensor by a user, the sensor further configured to measure a physical change upon receipt of a second voice command by the user, the physical change comprising information on the amount of fluid dispensed, and a wireless transmission module in communication with the sensor configured to communicate a physical change to a remote device upon receipt of the second voice command by the user. The method of dispensing and monitoring fluid dispensed comprises providing the fluid dispensing device described above, signaling by the user to the sensor the first voice command or the second voice command, wherein the sensor signals actuation of the fluid dispenser pump to dispense the fluid from the flexible and collapsible fluid container through the exit port in response to receiving the first voice command, and wherein the sensor wirelessly transmits information on the amount of fluid dispensed from the sensor to a remote device in response to receiving the second voice command by the end user.

In still another embodiment, the disclosure may be directed to an apparatus and method for dispensing and adjusting an amount of fluid dispensed. The apparatus comprises a flexible and collapsible fluid container, a fluid dispenser pump connected to the flexible and collapsible fluid container configured to draw fluid from the flexible and collapsible fluid container into the fluid dispenser pump and to dispense fluid through a fluid delivery channel to an exit port upon actuation of the pump, and a sensor located in the apparatus and configured to actuate the dispenser pump upon a first voice command received by the sensor by a user, the sensor further configure to adjust an amount of a dose of fluid to be dispensed upon receipt of a second voice command by the user. The method comprises providing the fluid dispensing apparatus described above, signaling by the user to the sensor the first voice command or the second voice command, wherein the sensor signals actuation of the fluid dispenser pump to dispense the fluid from the flexible and collapsible fluid container through the exit port in response to receiving the first voice command by the end user, and wherein the sensor to adjusts an amount of a dose of fluid to be dispensed in response to receiving the second voice command by the end user.

In another embodiment, the disclosure may be directed to an apparatus comprising a flexible and collapsible fluid container, a fluid dispenser pump connected to the flexible and collapsible fluid container configured to draw fluid from the flexible and collapsible fluid container into the fluid dispenser pump and to dispense fluid through a fluid delivery channel to an exit port upon actuation of the pump, and a biometric sensor located in the apparatus and configured to unlock actuation of the dispenser pump upon biometric identification information received by the sensor, the sensor further configure to unlock adjustment of an amount of a dose of fluid to be dispensed by the fluid dispenser pump upon biometric identification information received by the sensor. The method of unlocking the apparatus comprises providing the apparatus described above, detecting biometric identification information by the sensor, analyzing the biometric identification information to detect an initial biometric identification information, and in response to detecting the initial biometric identification information, allowing a user of the fluid dispensing device to actuate the fluid dispenser pump to dispense the fluid from the flexible and collapsible fluid container through the exit port, adjust the amount of the dose of fluid to be dispensed by said fluid dispenser pump, or both.

In a further embodiment, the disclosure may be directed to an apparatus for dispensing and monitoring fluid dispensed comprising a flexible and collapsible fluid container, a fluid dispenser pump connected to the flexible and collapsible fluid container configured to draw fluid from the flexible and collapsible fluid container into the fluid dispenser pump and to dispense fluid through a fluid delivery channel to an exit port upon actuation of the pump, the dispenser pump having an actuator comprising a dose delivery button which, upon depression by a user, actuates the fluid dispenser pump to dispense a dose of fluid, a flexible cover secured to an exterior surface of the container, wherein the flexible cover is of dimensions similar to dimensions of the container to protect the container, and wherein the flexible cover constructed of materials which are environmentally sustainable, a sensor located in the apparatus and configured to measure a physical change upon depression of the dose delivery button of the fluid dispenser pump by a user, the physical change comprising information on the amount of fluid dispensed, a wireless transmission module in communication with the sensor configured to communicate the physical change to a remote device depression of the dose delivery button of the fluid dispenser pump by the user, wherein the wireless transmission module communicates the physical change to the remote device only upon depression of the dose delivery button. The method comprises providing the flexible cover and dispensing device described above, and securing the flexible cover to an exterior surface of the container such that the flexible cover provides a protective surface to the container.

In another embodiment, the disclosure may be directed to an apparatus for dispensing and monitoring fluid dispensed comprising a flexible and collapsible fluid container, a fluid dispenser pump connected to the flexible and collapsible fluid container configured to draw fluid from the flexible and collapsible fluid container into the fluid dispenser pump and to dispense fluid through a fluid delivery channel to an exit port upon actuation of the pump, the dispenser pump having an actuator, and a cover secured to an exterior surface of the container, wherein the cover is of dimensions similar to dimensions of the container, the cover including sidewalls around a periphery of the flexible cover to encase the container therein, the cover constructed of materials which are environmentally sustainable, wherein the sidewalls of the cover include a flat end such that the container may be placed on an end thereof while on a substantially flat surface. The method comprises providing the cover and dispensing device described above, and securing the cover to an exterior surface of the container such that the flexible cover provides a protective surface to the container.

In yet another embodiment, the disclosure may be directed to an apparatus for dispensing and monitoring fluid dispensed comprising a flexible and collapsible fluid container, a fluid dispenser pump connected to the flexible and collapsible fluid container configured to draw fluid from the flexible and collapsible fluid container into the fluid dispenser pump and to dispense fluid through a fluid delivery channel to an exit port upon actuation of the pump, the dispenser pump having an actuator which actuates the fluid dispenser pump to dispense a dose of fluid, a sensor located in the apparatus and configured to measure a physical change upon actuation of the fluid dispenser pump by a user, the physical change comprising information on the amount of fluid dispensed, and a wireless transmission module in communication with the sensor configured to communicate the physical change to a remote device upon actuation of the fluid dispenser pump by the user, wherein the wireless transmission module communicates the physical change to the remote device only upon depression of the dose delivery button, and wherein actuation of the dispensing pump provides the sensor with dose information including dose amount, time of day, or both to predict the dose information on a future actuation of the dispensing pump transmitted to the remote device by the wireless transmission module. The method comprises providing the fluid dispensing apparatus described above, actuating, by the user, the fluid dispenser pump to dispense the fluid from the flexible and collapsible fluid container through the exit port, upon actuation of the fluid dispenser pump, sensing by the sensor information on the amount of fluid dispensed from the exit port, storing the information on the amount of fluid dispensed from the exit port, determining suggested dose information in response to the information on the amount of fluid dispensed from the exit port stored by the sensor, and providing the suggested dose information to the remote device by the wireless transmission module.

Accordingly, the embodiments provide a reliable and verifiable way for users of fluids to be automatically reminded to administer a dosage, for users to be sure that the appropriate dosage is being administered, and physician, pharmacy, retailers, manufacturers, or others requiring compliance, monitoring and reordering capabilities can reliably track and document usage.

Additionally, according to embodiments, there is provided a fluid dispensing device having a flexible actuator including a flexible dome and a flexible flange extension; a base portion including a bottom portion of a pump chamber, a pump chamber inlet, and a bottom portion of an outlet valve, said bottom portion of an outlet valve having an upper surface configured to mate with a bottom surface of said flexible flange extension; a dispensing head including a fluid outlet channel in fluid communication with said outlet valve, where the flexible actuator is mated to the base portion so that said flexible bulb and said bottom portion of a pump chamber form a pump chamber, and so that said flexible flange extension and said bottom portion of said outlet valve form an outlet valve; the fluid dispensing device also having an inlet valve positioned in proximity to said pump chamber inlet to selectively permit fluid flow through pump chamber inlet.

According to another embodiment, the fluid dispensing device is configured to dispense a volume of fluid that is less than the volume of said pump chamber.

According to another embodiment, the outlet valve of the fluid dispensing device permits the flow of fluid toward the fluid outlet channel during activation of the flexible dome, and toward the pump chamber when the flexible dome is released.

According to another embodiment, the inlet valve of the dispensing device initially permits fluid flow away from the pump chamber during activation of the flexible dome, and permits the flow of fluid into the pump chamber following release of the flexible dome. According to another embodiment, the inlet valve is formed as part of the flexible actuator. According to another embodiment, the inlet valve is attached to the base portion. According to another embodiment, the inlet valve comprises a flap integrally formed with said flexible actuator and a flexible film attached to said bottom part.

According to another embodiment, a flexible fluid pouch is provided having two openings, wherein a first opening is sealed around the flexible actuator of the dispensing assembly, and a second opening is sealed around the dispensing head of the dispensing assembly, and wherein the flexible flange extension and all of the bottom portion reside in the interior of said flexible fluid pouch.

According to another embodiment, the flexible pouch has only a single opening, and the single opening is sealed around the dispensing head of the dispensing assembly, and wherein the entirety of said dispensing assembly, i.e., the top piece comprising the flexible actuator and the bottom piece, reside in the interior of said flexible fluid pouch.

According to another embodiment, the fluid dispensing device defines a fluid flow path having an acute angle between the outlet valve and the fluid outlet channel.

According to another embodiment, the fluid dispensing device is configured so that increases in pressure on the flexible fluid pouch cause the outlet valve to seal more tightly against unintentional release of fluid.

### BRIEF DESCRIPTION OF THE DRAWINGS

The features of the embodiments are set forth with particularity in the appended claims. The figures are for illustration purposes only and are not drawn to scale. The embodiments, however, both as to organization and method of operation, may best be understood by reference to the detailed description which follows taken in conjunction with the accompanying drawings in which:

FIG. 1a is an exploded perspective view of an adjustable fluid dosing dispenser according to an embodiment.

FIG. 1b is an alternate rendition of the embodiment shown in FIG. 1a.

FIG. 1c is a perspective view of an adjustable fluid dosing dispenser dosing collar with an alternative slot design.

FIG. 1d is a bottom perspective view of the dosing dial/button.

FIG. 2 is a bottom perspective view of the dosing/control collar shown in FIG. 1a.

FIG. 3 is a cross-sectional view of the dosing/control collar shown in FIG. 1a.

FIG. 4 is a perspective view of a dosing/control collar according to a further alternative embodiment.

FIG. 5a is a perspective view of an assembled adjustable fluid dosing dispenser according to an embodiment in a closed/locked position.

FIG. 5b is an alternate rendition of the embodiment shown in FIG. 5a.

FIG. 6a is a perspective view of an adjustable fluid dosing dispenser according to another embodiment.

FIG. 6b is a side elevation view of the adjustable fluid dosing dispenser according to the embodiment of FIG. 6a.

FIG. 6c is a side cross-sectional view of the adjustable fluid dosing dispenser according to the embodiments of FIGS. 6a and 6b.

FIG. 7a is a perspective view of an adjustable fluid dosing dispenser according to an embodiment proximate to a matching opening in a flexible fluid container.

FIG. 7b is an alternate rendition of the embodiment shown in FIG. 7a.

FIG. 8a is a perspective view of an adjustable fluid dosing dispenser attached to the surface of a flexible fluid container according to an embodiment,

FIG. 8b is an alternate rendition of the embodiment shown in FIG. 8a.

FIG. 9 is a partial perspective view of an embodiment of the fluid dispensing device.

FIG. 10 is a perspective, partially exploded view of an embodiment of the fluid dispensing device.

FIG. 11 is a perspective view of an embodiment of the fluid dispensing device.

FIG. 12 is a perspective view of an embodiment of the fluid dispensing device.

FIG. 13 is a perspective view of an embodiment of the fluid dispensing device.

FIG. 14 is an exploded view of an embodiment of the fluid dispensing device.

FIG. 15 is a perspective view of an embodiment of the fluid dispensing device.

FIG. 16 is an exploded view of an embodiment of the fluid dispensing device.

FIG. 17 is a perspective view of an embodiment of the fluid dispensing device.

FIG. 18 is an exploded view of an embodiment of the fluid dispensing device.

FIG. 19 is a bottom perspective view of the embodiment of the fluid dispensing device shown in FIG. 18.

FIG. 20 is a perspective view of the embodiment of the fluid dispensing device shown in FIG. 18.

FIG. 21 is a perspective view of an embodiment of the fluid dispensing device.

FIG. 22 is a side plan view of the embodiment of the fluid dispensing device shown in FIG. 21.

FIG. 23 is a perspective view of the embodiment of the fluid dispensing device shown in FIG. 21.

FIG. 24 is a representative cutaway side view of an assembly according to an embodiment, at rest after priming.

FIG. 25 shows various external views of a dispensing assembly according to an embodiment.

FIG. 26 is a representative cutaway side view of the embodiment shown in FIG. 24, at a first stage of activation.

FIG. 27 is a representative cutaway side view of the embodiment shown in FIG. 24, at a second stage of activation.

FIG. 28 is a representative cutaway side view of the embodiment shown in FIG. 24, at a third stage of activation.

FIG. 29 is a representative cutaway side view of the embodiment shown in FIG. 24, at a first stage of recovery.

FIG. 30 is a representative cutaway side view of the embodiment shown in FIG. 24, at a second stage of recovery.

FIG. 31 is a representative cutaway side view of another embodiment,

FIG. 32 is a representative cutaway side view of the embodiment shown in FIG. 31, at a first stage of activation.

FIG. 33 is a representative cutaway side view of yet another embodiment.

FIG. 34 is a representative cutaway side view of a further embodiment.

FIG. 35 is a perspective view of still further embodiments in which all portions of the dispensing assembly except the head/neck/nozzle is situated inside the flexible pouch.

FIG. 36 is a side view of another embodiment in which all or nearly all of the dispensing assembly is situated inside the flexible pouch.

FIG. 37 is a top view of the embodiment shown in FIG. 36.

FIG. 38 is a perspective view of the dispensing assembly shown in FIGS. 36 and 37.

FIG. 39 is an exploded perspective view of the dispensing assembly shown in FIG. 38.

FIG. 40 is a cutaway perspective view of an embodiment.

### DETAILED DESCRIPTION OF THE EMBODIMENTS

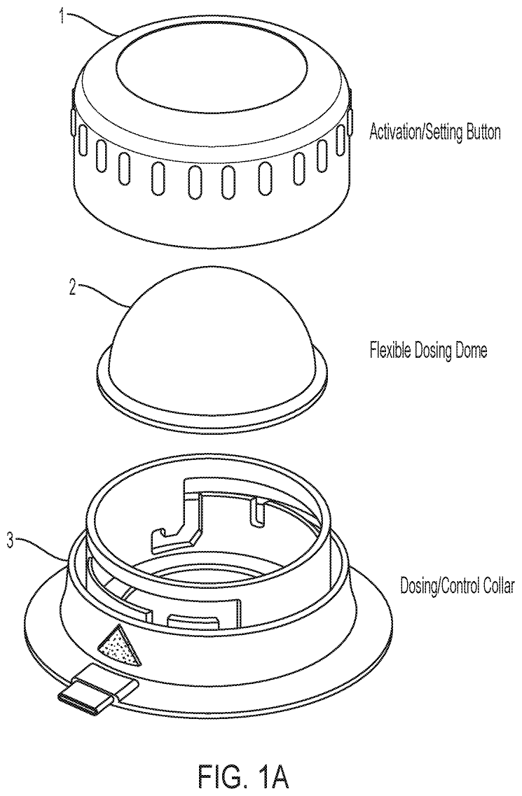

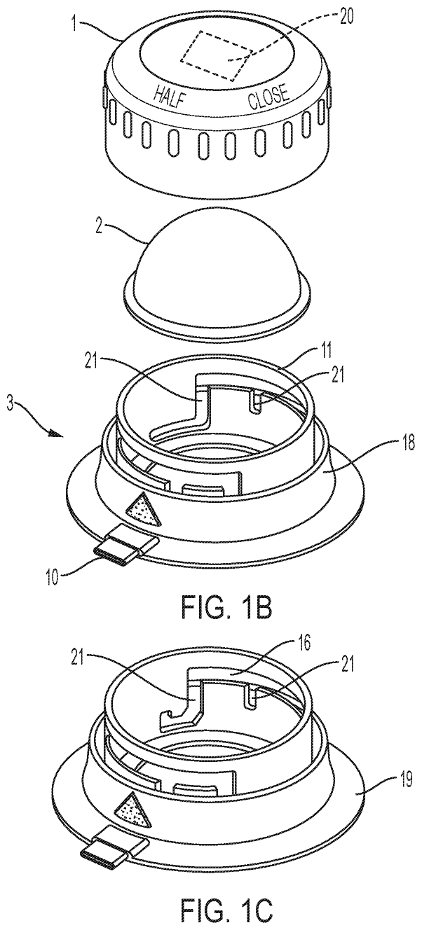

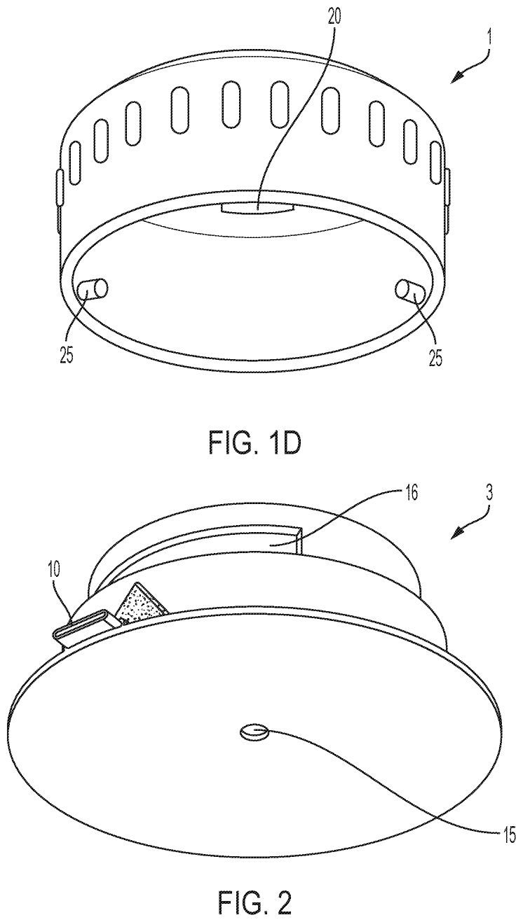

In describing the embodiments, reference will be made herein to FIGS. 1-23 of the drawings in which like numerals refer to like features of the embodiments.

The embodiments are directed to a device for dispensing fluid from a pouch, the device capable of being set to different discrete and repeatable/equal dispensing amounts, depending on the amount of fluid required to be dispensed by the user for various applications and uses. The device may also be set to an “off” or “closed” position to prevent accidental dispensing of fluid when not in use. The device is preferably configured to be affixed to the outer surface of a fluid container 50, which fluid container is preferably a flexible bag or pouch, though portions of dispensing device may be situated inside or outside the fluid container 50, provided that dosing dome 2 or button 1 is accessible from the outside of the flexible container for actuation by the user. According to various embodiments, at least one surface of the fluid container 50 or a sufficient portion of the fluid container 50 is flexible to allow the container to collapse as fluid is withdrawn therefrom. In the case that the entire fluid container 50 is not made of flexible material, the dispensing device is preferably attached to portion of the container 50 that is flexible and which is collapsible as fluid is dispensed from the container 50.

The device also includes one or more sensors 20 configured to record, store and/or transmit one or more physical properties of the device, particularly when the dispensing device is actuated in order to dispense fluid and/or when the device is adjusted to a particular dose.

FIGS. 1a-1e show an embodiment of the device including a dosing/control collar 3, a flexible dosing dome 2, and a combined dose setting dial and dose delivery button 1. The flexible dosing dome 2 and the dosing/control collar 3, when assembled, form the pump or dosing chamber. The dosing/control collar 3 is preferably cylindrical in shape with concentric inner and outer annular shafts/columns 11,18 rising from a common base and defining a narrow channel 17 between them. The exterior of the outer annular shaft 18 may feature a flange 19 that extends away from the center of the shaft. The interior shaft 11 also features a plurality of horizontal and vertical dosing and rotation channels or slots 21 that receive and interact with corresponding nubs on the outside surface of the dose dial/button 1. According to an alternative embodiment, the dosing and rotation channels may be on the shaft of the dose dial/button 1 and the nubs 25 may be on the outside surface of the inner annular shaft 11. The horizontal slots 16 are the rotation slots and may have a plurality of detente locations so that the user can tactically feel the progress of the dial as it is being rotated. The vertical channels 21 are the dosing slots and have differing depths (measured from the horizontal slot 16 to the bottom of the vertical slot) which correspond to different dispensing amounts. According to a preferred embodiment, each vertical slot 21 in the inner annular shaft 11 is paired with a second vertical slot 21 of the same depth, spaced apart on the inner annular shaft 11, and each nub 25 on the dose dial/button 1 is paired with a second nub 25 spaced apart on the dose dial/button 1 at a location that corresponds to the location of the second vertical shaft 21. When the dial/button 1 is rotated so that a nub 25 on the exterior of the dial/button Hines up with a vertical slot 21, the button 1 can be depressed to dispense fluid. The limit of depression limits the amount dispensed, and the depth of the vertical slot 21 limits how far the button 1 can be depressed because when the nub 25 hits the bottom of the vertical slot 21, the dial/button 1 is prevented from being depressed any further without breaking the nub 25, the slot/channel 21, or both.

According to one embodiment, there is at least one vertical slot or set of vertical slots 21 for a “Full” dose, and there is at least one second vertical slot or set of vertical slots 21 for a “Half” or “Partial” dose. According to other embodiments, there may be a third and fourth vertical slots or sets of vertical slots 21 for other fractional doses, for example, ¾ dose, ⅔ dose, ⅓ dose and ¼ dose.

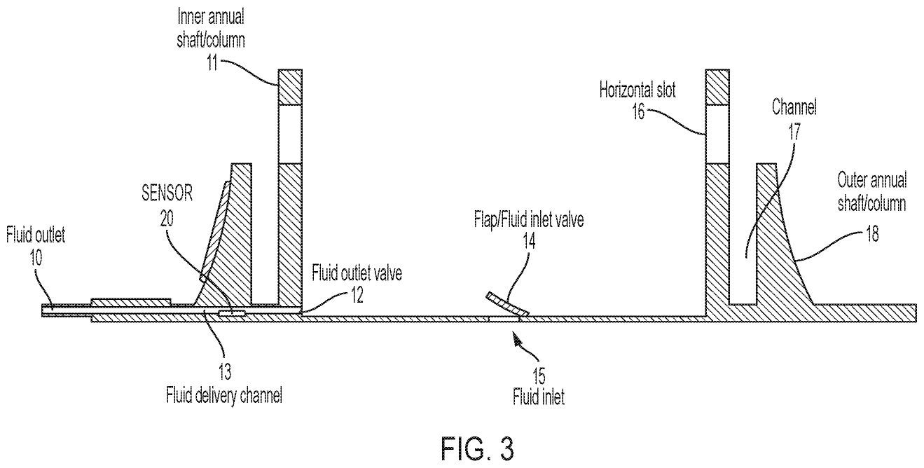

The dosing control collar 3 also features a through-opening 15 in the bottom surface to accommodate the entry of fluid, fluid inlet valve 14, represented in FIG. 3 as a flexible flap that is configured to lie over the through-opening, a fluid outlet 10 on the outside surface of the dosing control collar 3, and a fluid delivery channel 13 between said fluid inlet 15 and said fluid outlet 10.

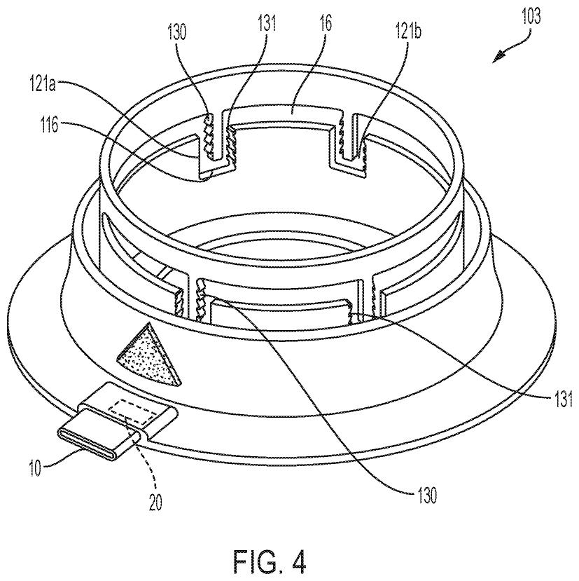

According to a further alternative embodiment shown in FIG. 4, the dosing collar 3 may have separate dosing slots 121a and return slots 121b. According to this embodiment, the dosing slots 121a may have downward facing teeth or prongs 130 which prevent the nubs 25 from traveling upwards, thus requiring that the dial/button 1 be fully depressed before it returns to the set position. Since the nubs 25 cannot travel upwards in the dosing slots 121a, separate return slots 121b are provided adjacent the dosing slots, connected by a horizontal slot 116. The return slots 121b preferably have upward facing teeth or prongs 131 to prevent the nubs 25 from traveling downward while in the return slots 121b.

The dose setting dial/dose delivery button is preferably manufactured of relatively rigid plastic having a rigid bottom portion that snaps into the channel 17 formed between the inner and outer annular shafts 11,18 of the base (dosing/control collar 3).

The dose setting dial/dose delivery button 1 and the dosing/control collar 3 are preferably made of a rigid plastic material. Nubs 25 molded to or otherwise formed on the interior surface of the dial/button 1 rest inside the slots formed in the interior shaft 11. When a user presses the dial/button 1, the dial/button 1 forces the flexible dosing dome 2 downward to evacuate the interior volume of the dome 2 via the fluid outlet 10; when the dial/button 1 is released, the flexible dosing dome 2 returns to its original shape, forcing the dial/button 1 upward, and drawing fluid into the interior of the dome 2 under vacuum action.

The flexible dosing dome 2 is preferably made of shape memory elastomeric material that returns to its original shape after deformation.

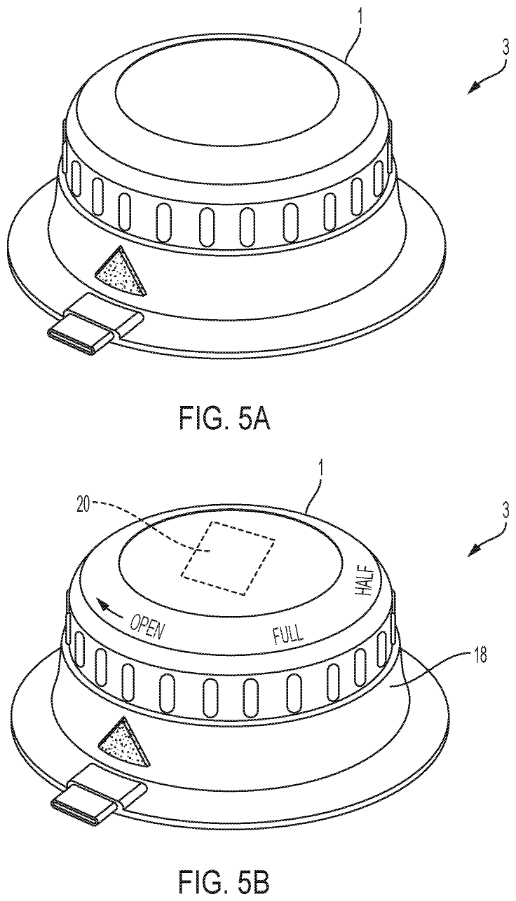

FIGS. 5a and 5b show these three parts assembled into an adjustable fluid dispensing device according to one or more embodiments in which the flexible dosing dome 2 is sealed to the base inside the interior column 11 of the dosing/control collar 3 and the bottom portion of the dose setting dial/dose delivery button 1 is snapped over the flexible dosing dome 2 into the channel 17 between the inner and outer shafts 1_1,18. The upper portion of the dial/button 1 projects above the top of the dosing/control collar 3. According to a preferred embodiment, the perimeter of the upper portion of the dial/button 1 has molded or printed indicia such as “Open” “Closed” and “FULL” and “HALF” to indicate the rotary position of the dial 1 that corresponds to various functions or dispensing amounts. Each of these indicia corresponds to a nub/slot combination that permits no depression, full depression, half depression, or other partial depression to dispense a corresponding amount of fluid.

Likewise, the outside surface of the outer shaft 18 of the collar 3 preferably has printed or molded or other indicia indicating the location on the collar 3 that must be lined up with the appropriate indicia on the dial/button 1 in order to achieve the desired function. In the configuration shown in FIGS. 5a and 5b, the dial/button 1 is in the fully depressed position, and rotated counterclockwise so that the nubs 25 on the inside surface of the dial/button 1 are in the bottom horizontal slot 30, locking the dial/button 1 into a closed position.

According to a preferred embodiment, the bottom horizontal slot 30 has a slight downward slant before becoming horizontal to draw the dial/button 1 down slightly as it is rotated into the closed position, see FIG. 1e, causing the bottom surface of the dial/button 1 to bear down on the fluid delivery channel 13, pinching it shut. As shown in FIGS. 5a and 5b, the indicia on the collar 3 lines up with a “<Open” indicia on the button 1, showing that in order to dispense fluid, the dial/button 1 must be rotated clockwise until the nubs line up with the “FULL” vertical slot. If the user wishes to dispense only a half dose, the dial/button 1 must be rotated clockwise again until the nubs 25 line up with the correspondingly shorter/shallower vertical channel/slot.

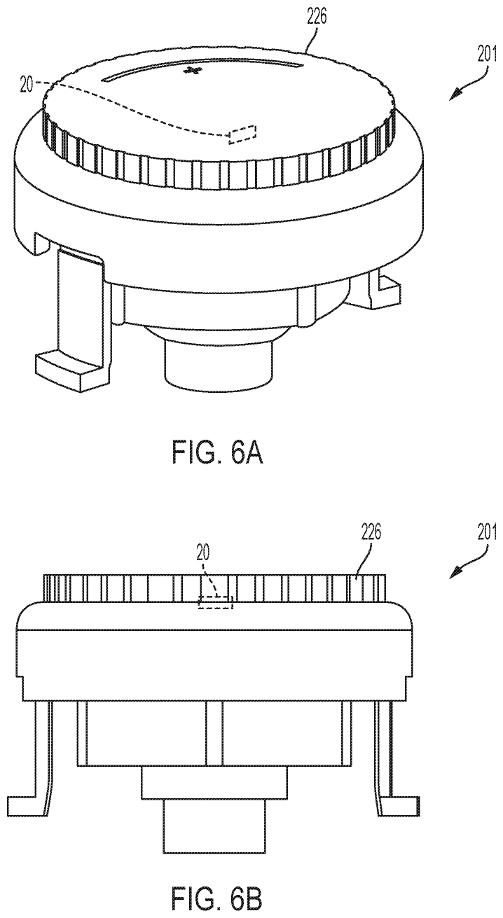

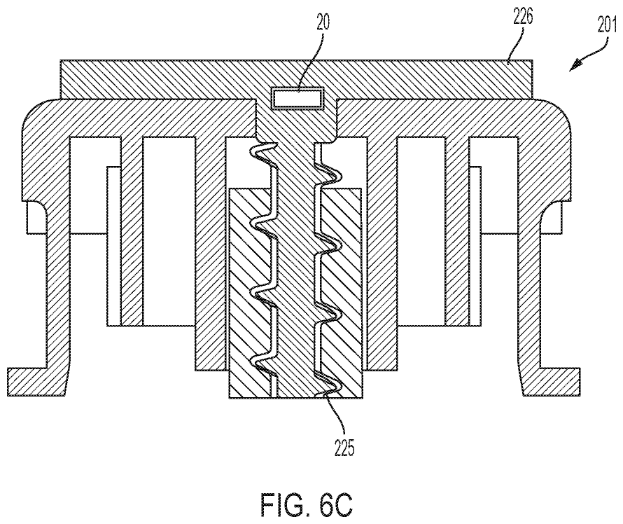

FIGS. 6a, 6b and 6c show an alternative design of an adjustable fluid dispensing device according to one or more embodiments. Instead of the nub and slot interaction of the device shown in FIGS. 1a-1d, 2, 4, 5a and 5b, the embodiment of FIGS. 6a, 6b and 6c contains a central screw 225 that can be turned by the top dial portion 201 for a continuous dose adjustment device. As the screw 225 is turned, the maximum depression of the button 201 of increased or decreased as the screw 225 moves the button 201 toward or away from the dosing control collar 3. As with the embodiment of FIGS. 1a-1d, 2, 4, 5a and 5b, a sensor 20 or other smart chip may be arranged to detect the rotation and hence the dosage amount.

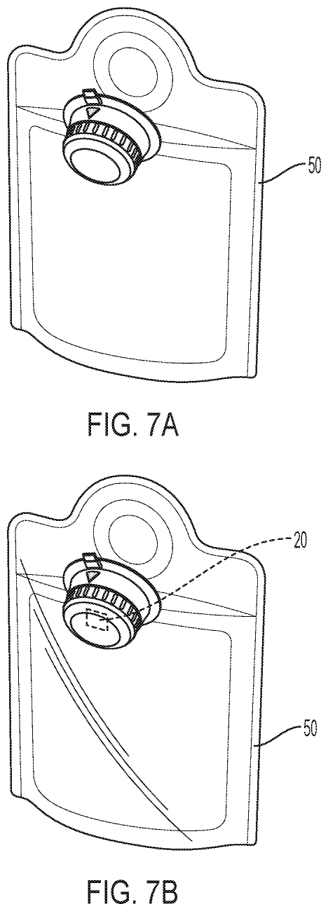

FIGS. 7a and 7b show the adjustable metering device of one or more embodiments about to be connected to a flexible fluid container 50, preferably a bag or pouch. According to a preferred embodiment, the fluid container 50 is collapsible as fluid is dispensed therefrom and preferably contains no air. According to one embodiment, the bottom of the adjustable metering device has an adhesive that makes a secure and air-tight connection to the fluid container 50. According to a further embodiment, the adhesive may be covered prior to use with a thin pull-away film to protect and preserve the adhesive until it is time to connect the device to the fluid container 50. According to various alternative embodiments, the bottom of the adjustable metering device may be heat welded to the fluid container 50, sonic welded to the fluid container 50, or sealed to the fluid container 50 in any other known method. According to a further embodiment, the fluid container 50 may have a reinforced region and/or treated surface that corresponds to the shape and size of the device to facilitate strong and secure connection and prevent container breaking or tearing should a force or load be applied to the device after it has been attached to the fluid container 50.

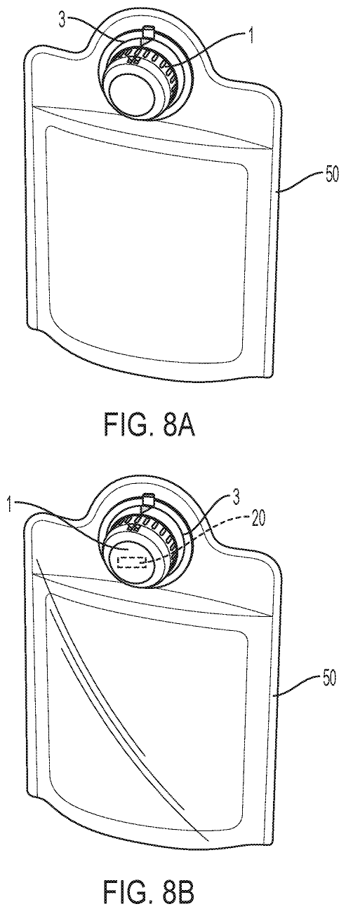

FIGS. 8a and 8b show an adjustable metering device of one or more embodiments connected to a flexible fluid container 50. The device can be operated to dispense fluid no matter the orientation of the device in space, as it is agnostic to gravity or other forces except for the depression of the dial/button 1.

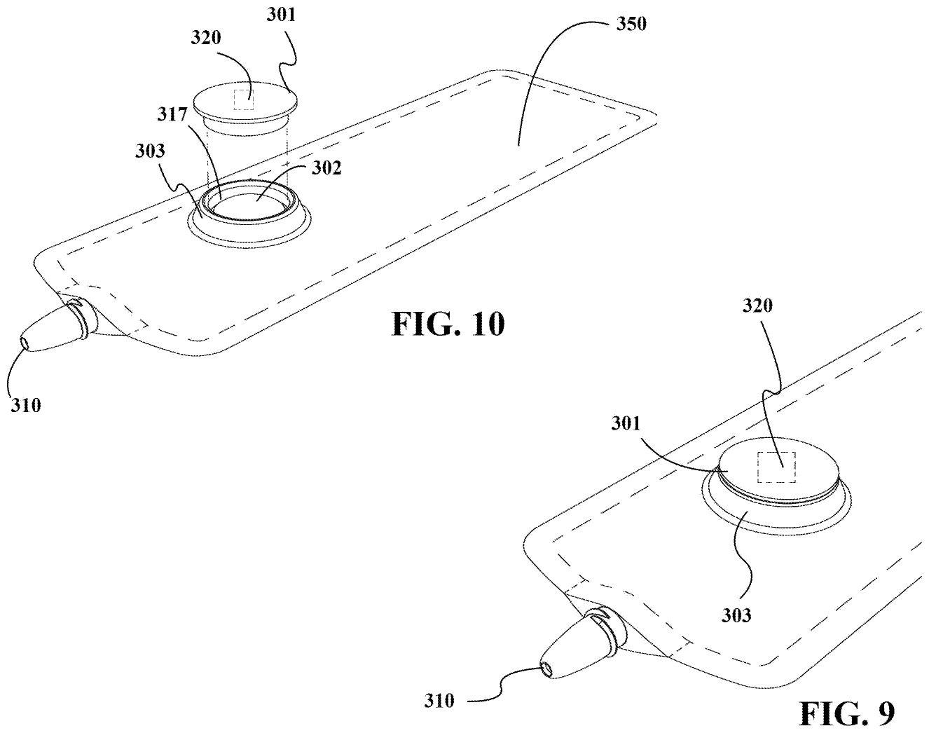

Referring now to FIGS. 9-10, further embodiments are shown, wherein an adjustable fluid dispensing device may be dispensed without physical depression of the flexible dosing dome. In FIGS. 9-10, a non-mechanical, digital fluid dispensing device for dispensing fluid is shown. Unlike the dispensing device of the previous embodiments, dispensing of a fluid is accomplished using a touch sensor 320 within button 301, which may be, for example, a 5-wire resistive touch sensor, a surface capacitive touch sensor, a projective capacitive touch sensor, a surface acoustic wave sensor, an infrared touch technology sensor, or any other touch sensor known in the art. Upon physical contact with button 301, touch sensor 320 signals movement of the flexible dosing dome 302 downward to evacuate the interior volume of the dome 302 via the fluid outlet 310 and subsequently allows the flexible dosing dome 302 to return to its original shape, thereby drawing fluid into the interior of the dome 302 under vacuum action. Thus, the fluid dispensing device of the present embodiment requires less force to dispense fluid than the mechanical fluid dispensing device of the previous embodiment. In some embodiments, the digital fluid dispensing device also requires no physical depression of the button 301 against the flexible dosing dome 302 to reload or ready the fluid dispensing device of the present embodiment. Button 301 may be of integral construction with dosing control collar 303 as shown in FIG. 9 or may be of separate construction as depicted in FIG. 10, where the button 301 includes an annular projection 325 which is complimentarily fit within channel 317 formed between collar 303 and dosing dome 302, so as to be removeable or replaceable.

In either of the digital embodiments depicted in FIGS. 9-10, the fluid dispensing device may include further digital features, which may include use touch sensor 320 to select the maximum depression of dosing dome 302. In these embodiments, button 301 may include a GUI in which a user may select incremental amounts of doses, which may include a half or partial dose. After making a selection, the sensor 320 signals a maximum depression of the dosing dome 302 to deliver a dose which corresponds to the selection made by an end user and is less than a single dose of fluid.

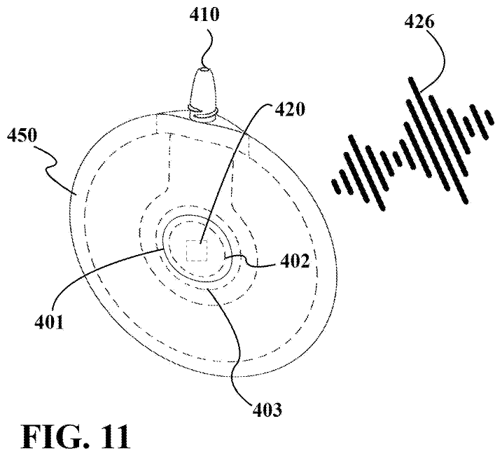

In some embodiments, the actuation of the digital dispensing device is voice-activated, as shown in FIG. 11. In these embodiments, one or more sensors 420 may comprise a sound sensor, for example an in-built capacitive microphone, a peak detector and an amplifier, or other similar devices known in the art. Sound sensor 420 is designed to respond to voice commands 426 to actuate the fluid dispensing device, wherein the dose within the interior of dome 402 is evacuated, delivered via the fluid outlet 410, and a new dose is readied within the interior of the dome 402. In these embodiments, the sound sensor 420 may be capable of recording specific voice commands 426 which are then required to be received to sound sensor 420 before a dose may be dispensed. In some embodiments, voice commands 426 may also change the volume of the dose delivered via fluid outlet 410 and/or transmit information regarding the dose or amount of fluid within 450 to one or more devices wirelessly for the purposes of tracking dose information, reporting malfunctions/proper operation of the fluid dispensing device, or reordering. The sound sensor 420 may require the voice commands 426 to be of a specific frequency in order to prevent misuse of the fluid dispensing device or accidental/premature dispensing of a dose within the fluid dispensing device.

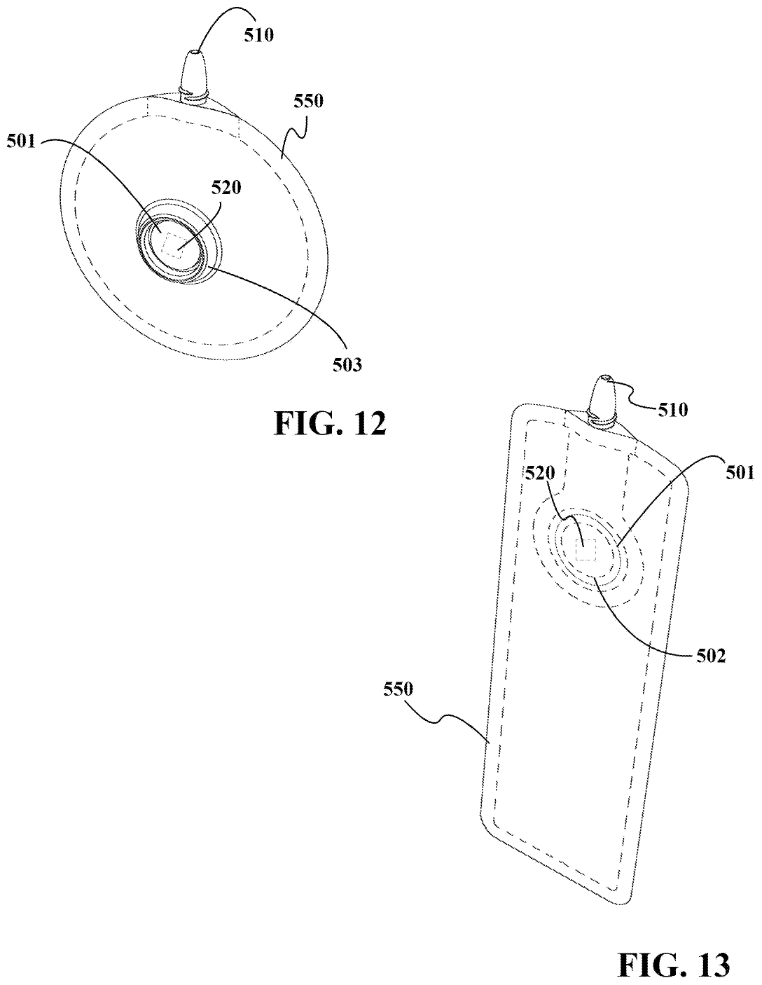

In an embodiment depicted in FIGS. 12 and 13, the fluid dispensing device may include a sensor which may be utilized for adjusting, dispensing, or readying a metered volume of fluid to be dispensed through a biometric identification and/or administration. In these embodiments, the biometric sensor 520 is designed to detect an end user through facial recognition, fingerprint recognition, or any other biometric sensing technology which is known in the art. The fluid dispensing device of the present embodiment may be mechanically actuated through depression of the button 501 only upon the biometric validation described above. In the event that the biometric validation procedure is not met, the fluid dispensing device will remain inoperable, and depression of the button 501 will not dispense a dose. Alternatively, the fluid dispensing device may dispense a dose of fluid automatically or digitally without any mechanical depression. In such embodiments, after biometric validation procedures are met by biometric sensor 520, the procedures to operate fluid dispensing device begin, thereby causing evacuation of flexible dosing dome (not shown), delivery of a dose via the fluid outlet 510, and reloading of a dose by readying flexible dosing dome.

In any of the above embodiments, one or more components of the fluid dispensing device may be constructed of sustainable, or organic-based materials. These sustainable materials may cardboard, paper, wood, compostable and biodegradable plastic alternatives, or any other similar of any suitable non-plastic polymer which may be of rigid or semi-rigid construction, such as cardboard, paper, wood, cornstarch, mycelium, agar, cotton, tapioca or any similar compostable and/or biodegradable material known in the art. These sustainable materials are suitable for use in lamination, layering, or reacting with other rigid, or semi-rigid materials for optimum functional, or tactile operation of the fluid dispensing device.

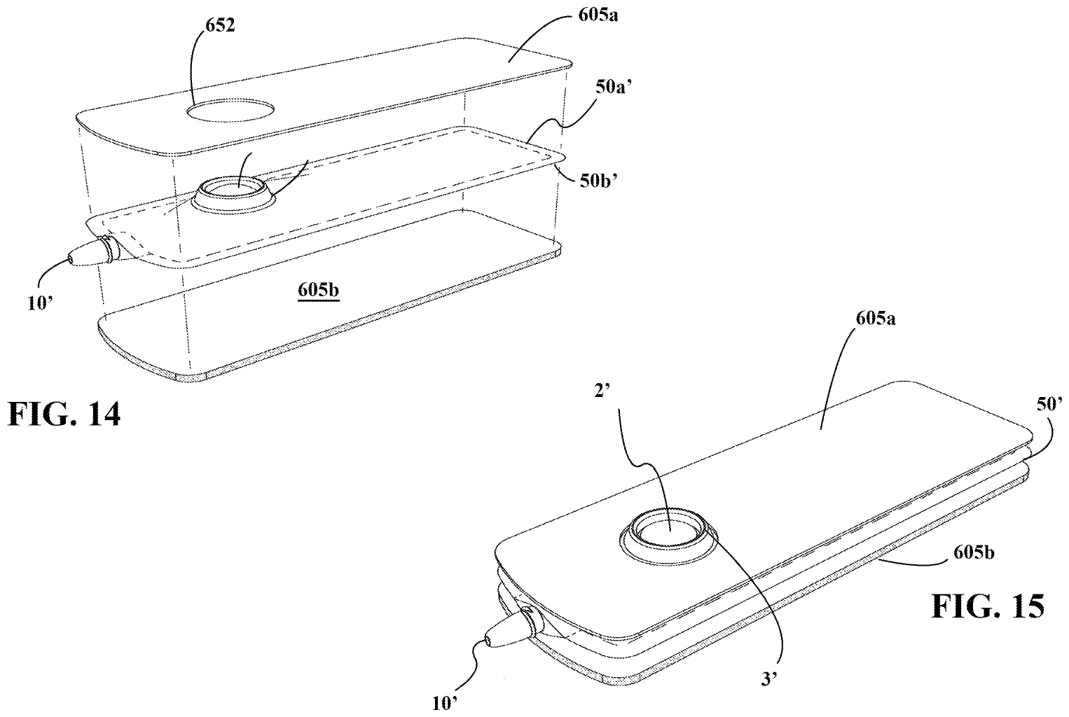

In another embodiment depicted in FIGS. 14-23, the mechanical and digital fluid dispensing devices of the previous embodiments may contain include the sustainable materials on the exterior of the flexible container, thereby providing additional protection to flexible container. In an embodiment shown in FIGS. 14 and 15, the fluid dispensing device of one or more of the previous embodiments is shown, comprising a substantially rectangular pouch 50′ having exposed on an upper surface the upper portion of dispensing collar 3′ and dosing dome 2′. Dispensing port or nozzle 10′ is connected to outlet channel (not shown) to dispense the fluid contents of the flexible container 50′ in a metered fashion. Exterior covers of substantially the same dimensions as the flexible container are attached to the upper and lower surfaces of flexible container 50a′, 50b′, respectively. Cover 605a includes an opening 652 sized to permit at least dosing dome 2′ to be accessed from the exterior. Covers 605a, b may be made of soft and partially flexible sustainable materials that are more rigid than the layers used for flexible container 50′, but still sufficient to bend, for example, when placed in a user's pocket. During assembly, heat or any suitable adhesive may be used to laminate or otherwise secure exterior covers 605a, 605b to the flexible container. The sides of the flexible container 50′ may remain exposed, including the end for dispensing nozzle 10′. Alternatively, only one of exterior cover 605a or 605b may be used, and the other side of flexible container 50′ left exposed, or one or both of exterior covers 605a, 605b may cover less than all of a side of the flexible container 50′.

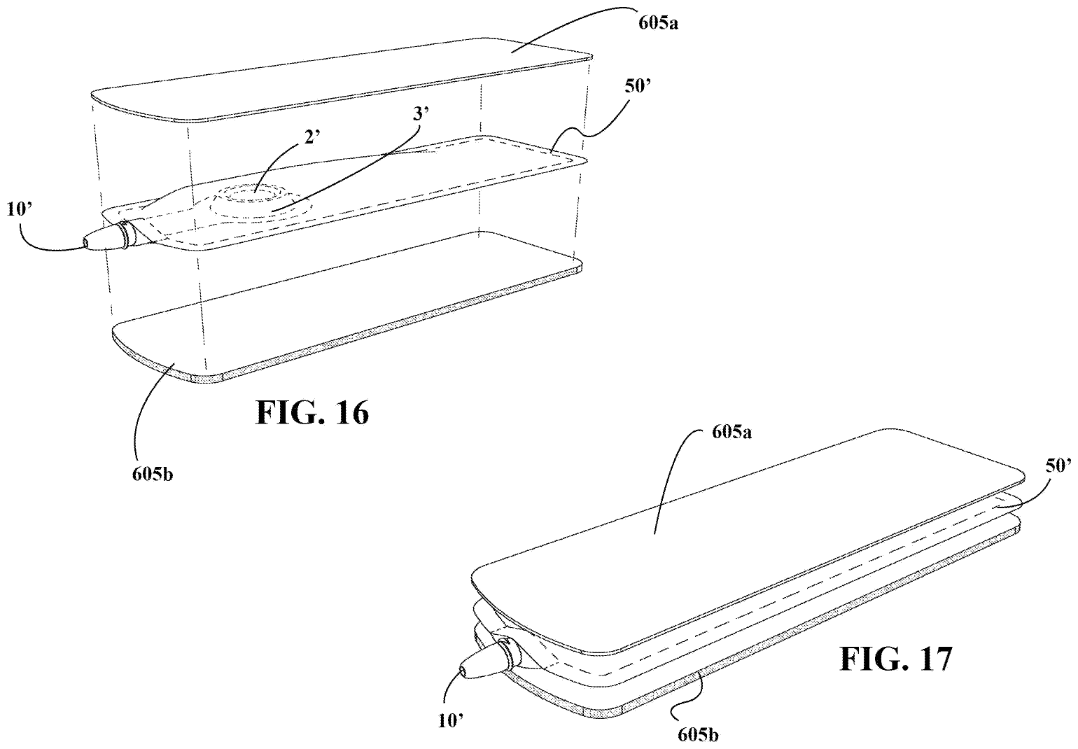

An alternative to the soft and partially flexible covers of FIGS. 14-15 is shown in FIGS. 16-17, where both covers 605a,b have no openings therein. However, cover 605a may still sufficiently flexible to permit the user to push through the cover at a marked location and depress dome build 102 thereunder. In other embodiments utilizing the digital dispensing device of the previous embodiments, cover 605a may be include the touch sensor, biometric sensor, or sound sensor (not shown) of the previous embodiments to allow dosing of the fluid dispensing device without physical depression.

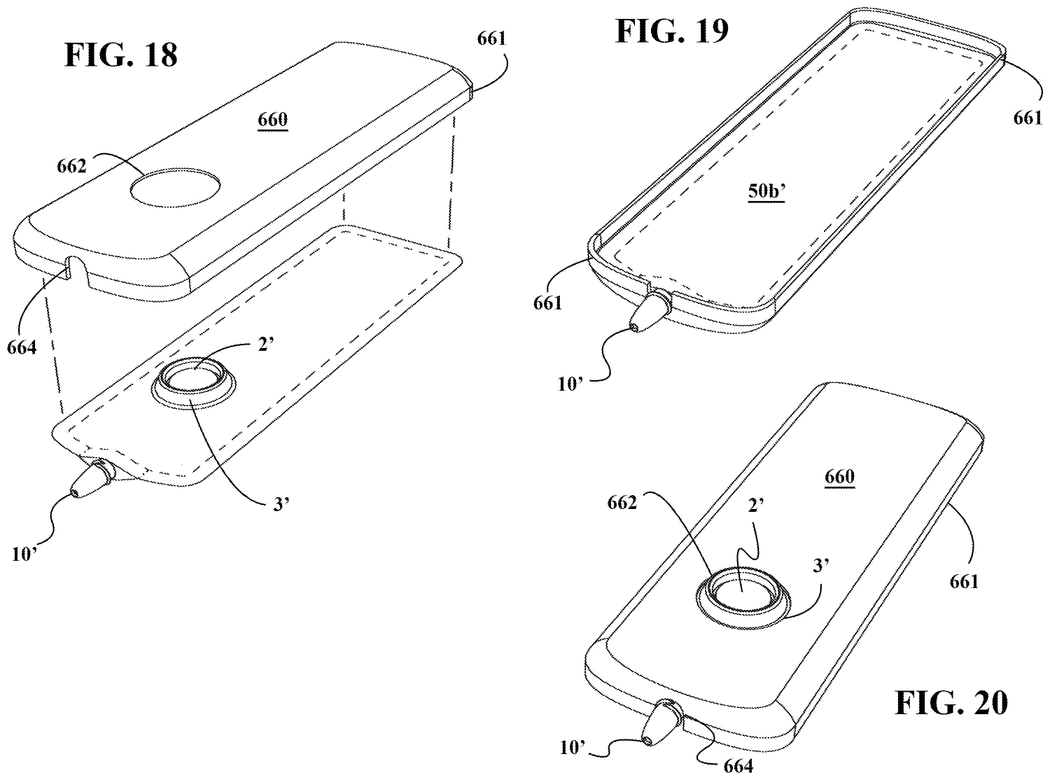

Instead of these soft and partially flexible covers, the present embodiments may utilize rigid or semi-rigid cases as shown in FIGS. 18-20. In FIG. 18, an open-bottom case 660a made of sustainable materials having more rigidity than the previous covers is fitted over the upper side of the flexible container 50a′. Case 660a has opening 662 aligned with collar 3′ and dosing dome 2′. Side walls 661 are provided around the periphery to cover the sides edges of container 50′, except for notch 664 for nozzle 10′. After assembly, the flexible container 50′ is secured to and nested within cover 660a, as shown in the inverted view of FIG. 19. In the assembled view of FIG. 20, the upper portion of dispensing collar 3′ and dosing dome 2′ are exposed and extend through opening 662, and nozzle 10′ extends through notch 664.

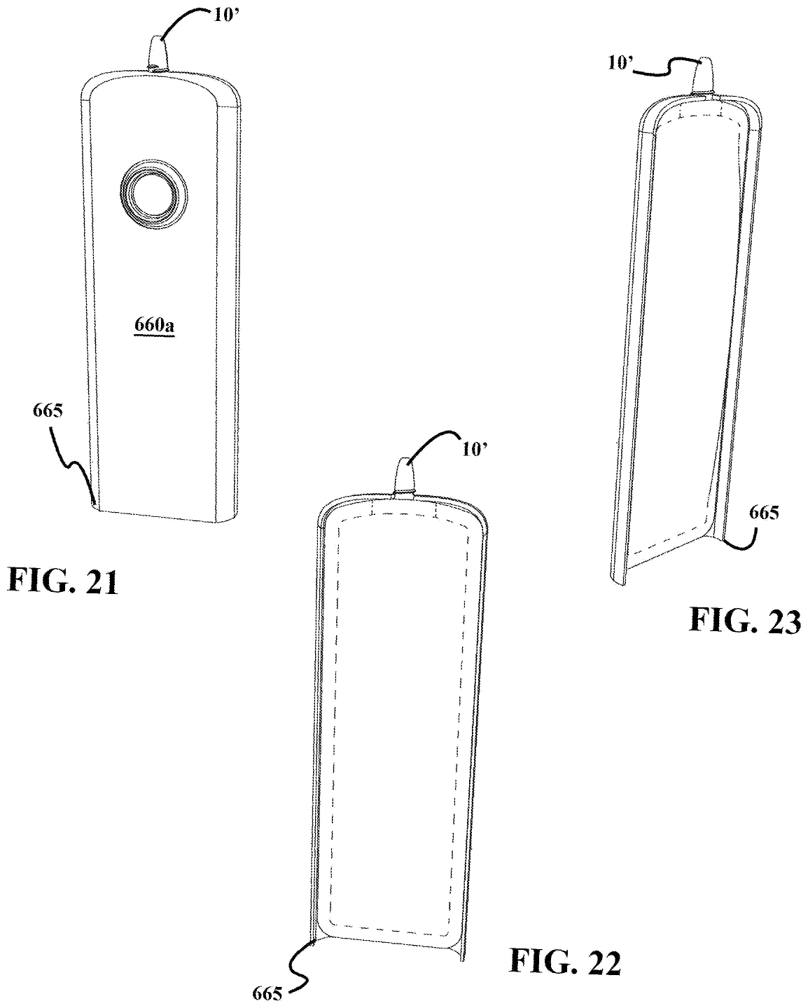

Any of the covers 605a, 605b or case 660a may be configured with flat, squared-off ends so that the covered or encased container 50′ may be stood up on end on top of a flat surface, such as a table or shelf. For example, in FIGS. 21-23, case 660a has flat end 665 which permits the fluid dispensing device to be placed in a stable upright position with the dispensing nozzle 10′ pointed upwards, for user functionality and storage.

While the covers 605a, 605b and cases 660a are shown configured for a substantially rectangular pouch 50′ in FIGS. 14-23, they may be configured to correspond to and fit containers of different configurations, such as circular or triangular.

In any of the previous embodiments, the sensor 20 or smart chip of the fluid dispensing device may be used to learn and anticipate the dispensing features of the previous embodiments. In these embodiments, the sensor 20 may utilize artificial intelligence analysis and feedback to predict the timing of fluid dosing, the amount of fluid dosing, or other performance tasks of the previous embodiments. In one example, the sensor 20 may record or transmit wirelessly tracked data information based on the habits and/or performances of the fluid dispensing device to set reminders, forecast functionality, and/or communicates next steps, which could include reordering, location prediction to facilitate delivery, or other functionalities which may be necessary.

Application will represent educational materials on the fluid being dispensed.

Compliance measured by actual pressure on the button 1 and number of times pushed. Time and date captured. Ability to reorder when pouch is close to deletion.

Smartphone application automatically track the dose size and time using either the mechanical or sensor technologies.

Data can be shared with physician, pharmacy, retailers, manufacturers or others, as permissible by users, requiring compliance, monitoring and reordering capabilities. Sensor 20 and app technologies allow for identification of reorder options using GPS technologies or online ordering applications.

According to an embodiment the fluid can be of any viscosity.

According to another embodiment RFID, sensor stickers, Bluetooth and other sensor technologies are used.

The user will download the app. The app will pick up the sensor signal from the sensor technology and prompt for educational and compliance engagement.

The app will automatically capture dose, time of dose, prompt for next dose, and alert when near depletion, requiring a reorder.

This is doable from day one using readily available sensor technologies.

The app will alert when the dose is due.

Pressure sensor 20 in the “button” sends a signal that the fluid was dispensed and how much. 1 TOUCH dispensing technology indicates dosage.

Patient can provide permissions through the app to share with physician, pharmacy, retailers, manufacturers or others requiring compliance, monitoring and reordering capabilities.

Sensor technologies provide automatic tracking to ensure ease of use. GPS enables real-time capabilities for reorder and retail opportunities.

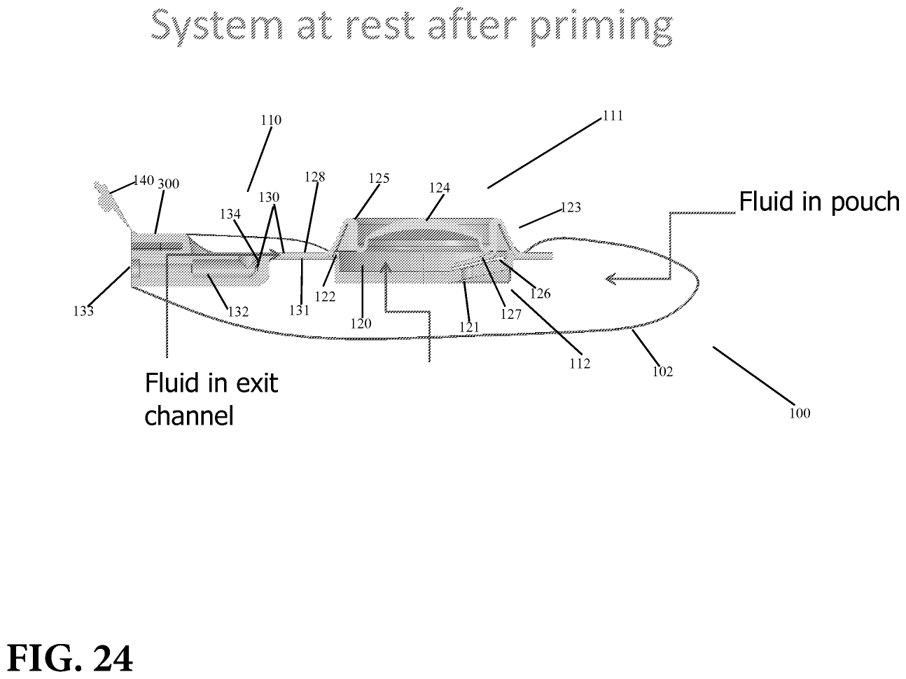

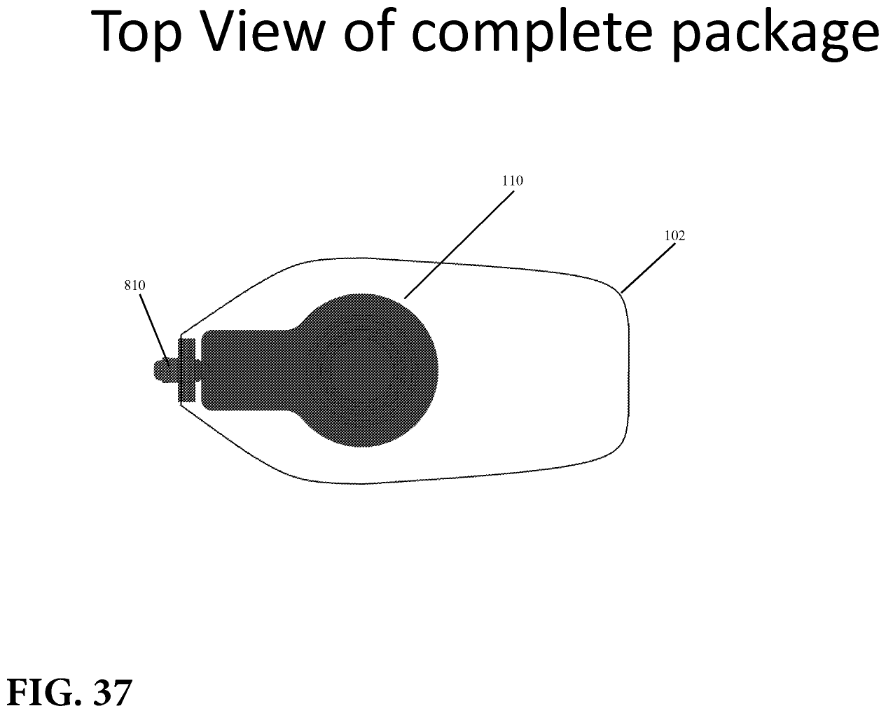

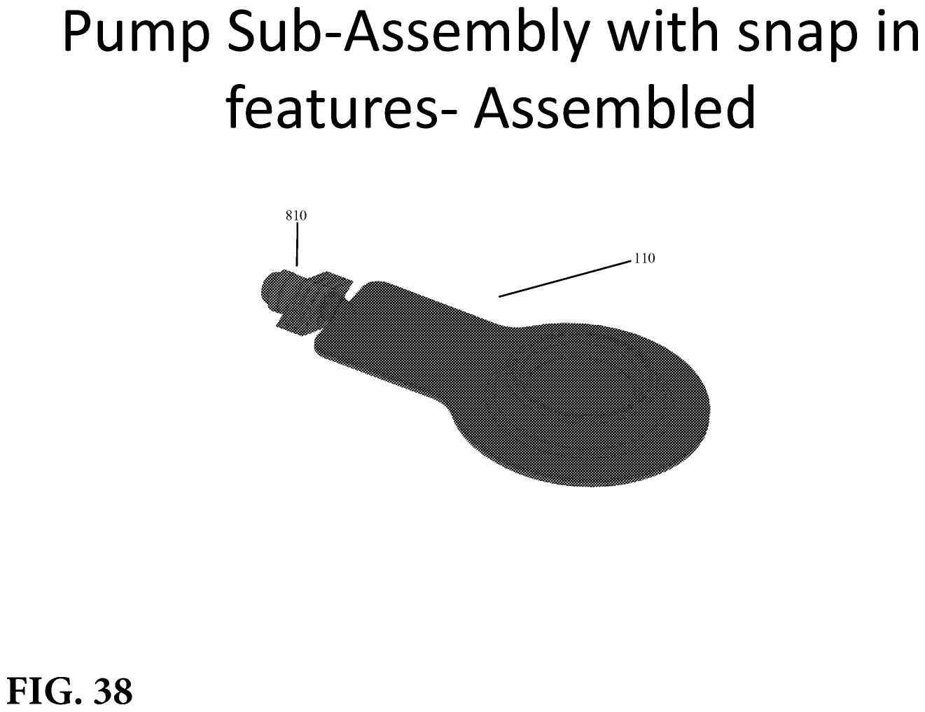

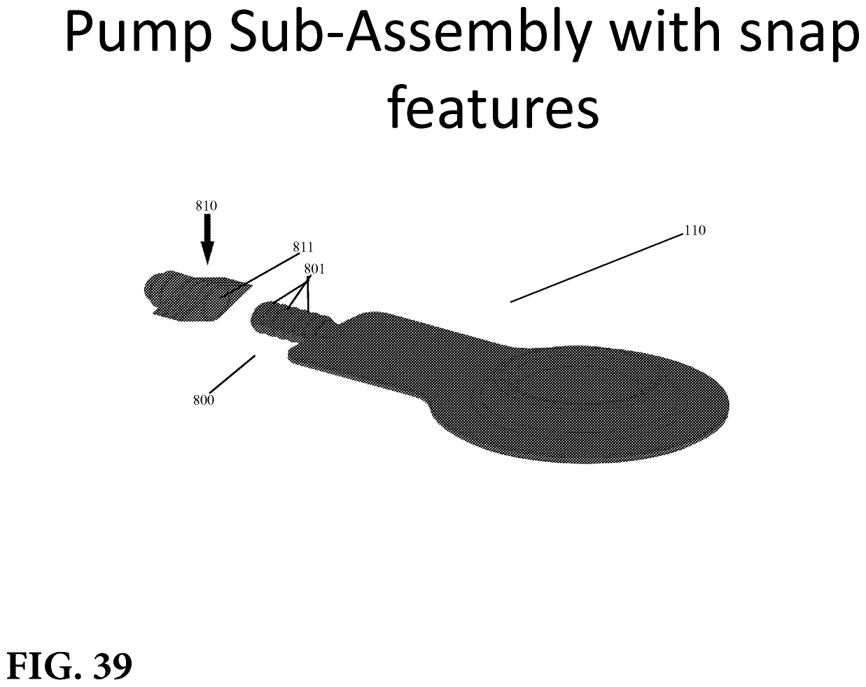

Additional nonlimiting features of the pump or dosing chamber that may be utilized for the fluid dispensing device of the embodiments shown in FIGS. 10-23 (at least) will now be addressed with reference to FIGS. 24-40. To the extent reference numbers in the discussion and illustration in FIGS. 24-40 overlap with reference numbers discussed above and illustrated in FIGS. 1-23, such overlapping is not intended to imply the same feature or element is being referenced.

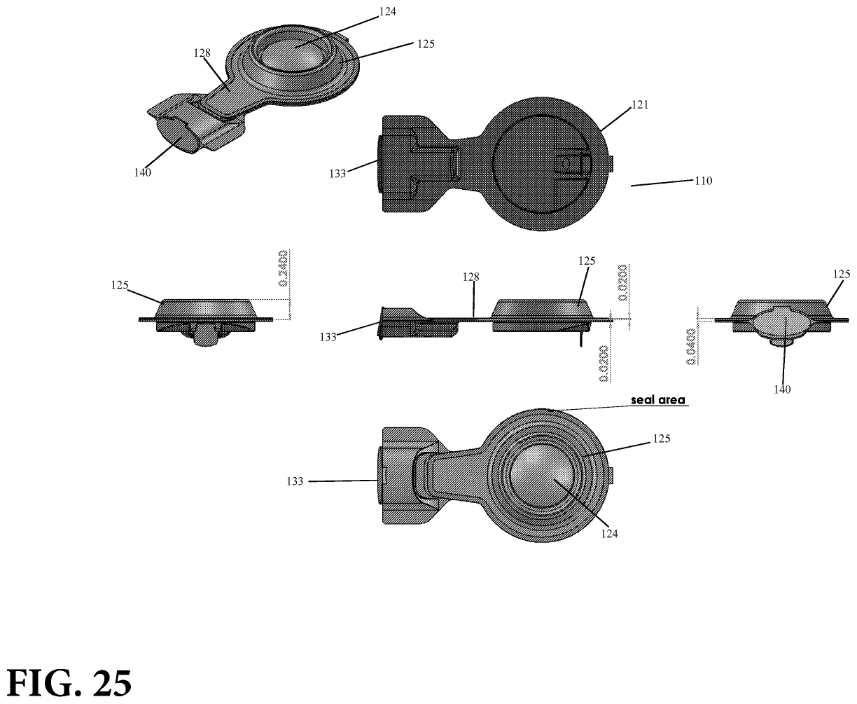

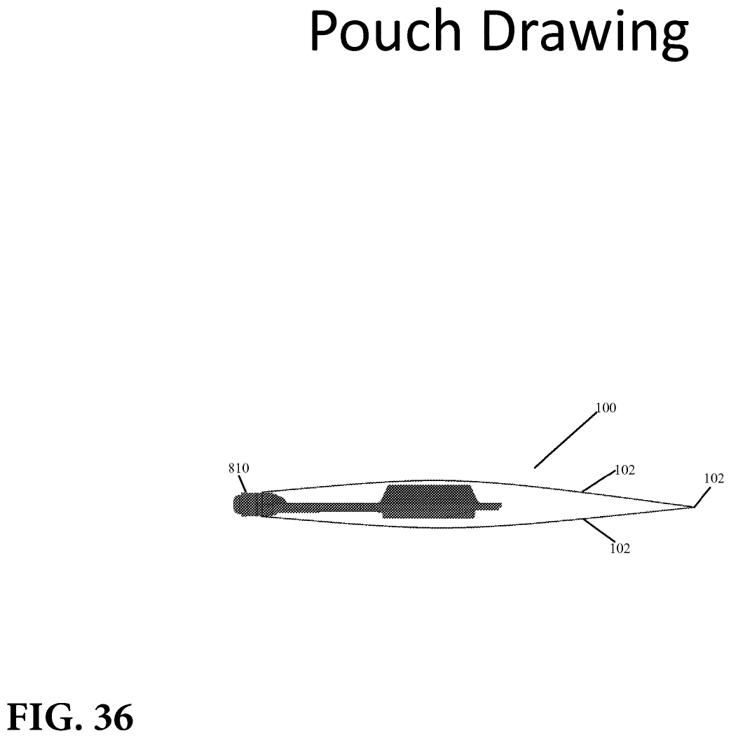

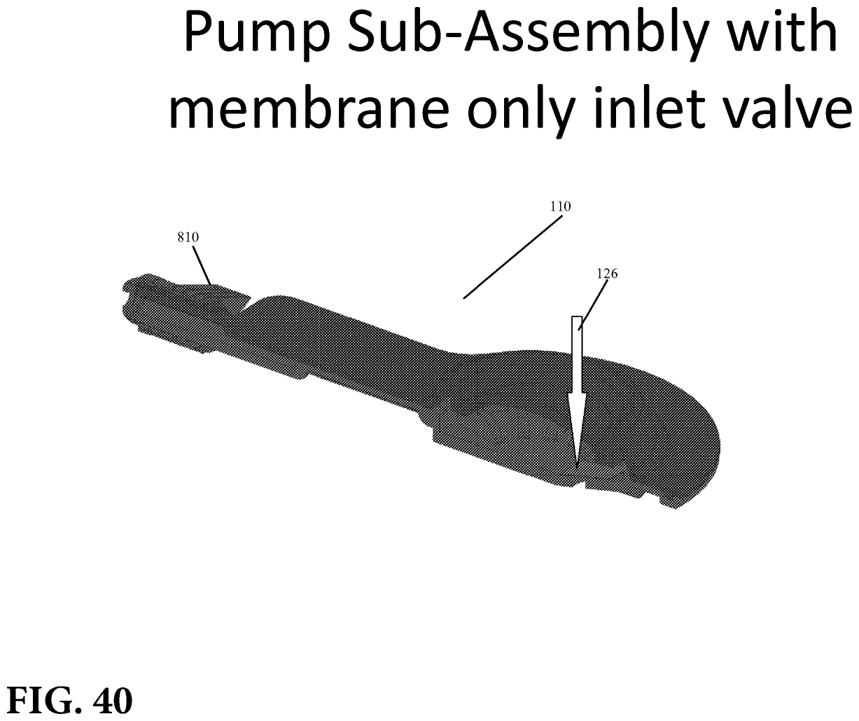

Referring to FIG. 24, a fluid dispenser (shown generally at 100) is provided having a flexible pouch 102 containing a fluid therein that is to be dispensed. A dispensing assembly (shown generally at 110) is provided in fluid communication with an interior of flexible pouch 102, and is sealed to the flexible pouch around the perimeter of dispensing assembly 110. More specifically, flexible pouch 102 may be provided with two openings, one opening sized to fit around and sealed to the perimeter of flexible actuator 123, and another opening sized to fit around and be sealed to neck 300. According to this embodiment, most of the dispensing assembly is situated inside the flexible pouch, and the only portion of the dispensing assembly that resides outside the flexible pouch is flexible actuator 123, and possibly a portion of neck 300. Dispensing assembly is preferably made of two molded pieces, top piece 111 and bottom piece 112 which are snapped and/or sealed together, and which preferably have male-female mating faces to improve fit/matching registration. Generally, top piece 111 is shown in the figures, and bottom piece 112 is shown (except FIG. 25, in which bottom piece 112 is shown, and FIGS. 36-40, in which bottom piece 112 is shown). According to various embodiments, top piece 111 may comprise only the flexible actuator 123 (including bulb 124 and circumferential wall 125), the flange, the flexible flange extension 128, and optionally the pump chamber inlet closure 127, and bottom piece 112 may comprise the remainder of the assembly, including the bottom portion of chamber 120, pump chamber inlet 121, the bottom portion of intermediate channel 131, all of outlet channel 132 and all of neck 300. According to other embodiments, the outlet channel 123 and the neck 300 (also referred to as head 800 in various figures), may be formed by a mating of the top part 111 to the bottom part 112, see, e.g., FIG. 40.

In use, a user operates dispensing assembly 110 to draw fluid from inside of flexible pouch 102 and to dispense an amount of the fluid that has been drawn into dispensing assembly 110, all as further described below.

Dispensing assembly 110 includes a pump chamber 120 and a fluid exit channel 130 in fluid communication with the pump chamber 120. As discussed in further detail below, pump chamber 120 serves as an intermediate chamber to pull a volume of fluid into dispensing assembly 110, and fluid exit channel 130 serves to deliver a portion of such volume of fluid from pump chamber 120 to the outside of fluid dispenser 100.

Pump chamber 120 includes a pump chamber inlet 121 that opens at one end to an interior of flexible pouch 102, and opens at the opposite end into the interior of pump chamber 120. Ultimately, a portion of the fluid that is drawn into the interior of pump chamber 120 from the interior of flexible pouch 102 is delivered to fluid exit channel 130 through fluid outlet 122 from pump chamber 120.

In order to draw fluid into pump chamber 120 and to push a portion of such fluid downstream to fluid exit channel 130, a flexible actuator 123 is provided having a depressible bulb 124 that may be engaged by a user's finger when they wish to dispense a volume of fluid from fluid dispensing 100, which dispensing operation will be discussed in greater detail below. Flexible actuator 123 may be formed of a variety of flexible materials, such as (by way of non-limiting example) a thermoplastic polyolefin elastomer. A suitable and exemplary thermoplastic polyolefin elastomer material is readily commercially available from The Dow Chemical Company under the name ENGAGE™ 8401. Flexible actuator 123 also includes a raised circumferential wall 125 that encircles depressible bulb 124, with a bottom-most portion of depressible bulb 124 being attached to a bottom, interior edge of circumferential wall 125. When a user engages flexible actuator to dispense a volume of fluid from fluid dispenser 100, depressible bulb 124 deforms from a concave structure to a convex structure (as it is pushed downward into the body of pump chamber 120), while raised circumferential wall 125 generally maintains its shape.

A thin cover 126 may optionally be positioned over pump chamber inlet 121, and is preferably formed of a thin, flexible film that is affixed at one end to an interior of pump chamber 120, and is not fixed at the other end which is in turn positioned over pump chamber inlet 121. Thus, thin cover 126 may freely move with respect to pump chamber inlet 121 to allow fluid to flow from the interior of pouch 102 to the interior of pump chamber 120, and likewise from pump chamber 120 back to the interior of pouch 102, depending upon the position of flexible actuator 123 (as discussed in greater detail below).

Alternatively, or in addition to thin cover 126, a pump chamber inlet closure 127 may be provided and positioned over pump chamber inlet 121, movable from an open position (allowing fluid to flow from the interior of flexible pouch 102 to an interior of pump chamber 120, and vice versa) to a closed position (preventing fluid from flowing from the interior of flexible pouch 102 to an interior of pump chamber 120, and vice versa). Pump chamber inlet closure 127 is attached at one end to preferably the bottom-most portion of circumferential wall 125, which point of attachment creates a hinge allowing the remaining portion of closure 127 to move between its open and closed positions. Pump chamber inlet closure is preferably formed of the same material as flexible actuator 123, and is likewise preferably integrally formed therewith. Preferably, in an at-rest position, but after a user has at least initially pumped flexible actuator 123 so as to prime dispensing assembly 110 with a volume of fluid, a slight gap exists between the bottom surface of pump chamber inlet closure 127 and the top of pump chamber inlet 121. Likewise, when at rest, a very slight gap between such thin film inlet cover 126 and the top of pump chamber inlet 121—only a slight gap is necessary at this stage to allow bi-directional flow between the interior of pump chamber 120 and the interior of flexible pouch 102. Pump chamber inlet closure 127 is configured to (as discussed in greater detail below) flex downwardly and press against thin film inlet cover 126 (if present) at a point during the downward stroke of flexible actuator 123, thus pressing itself against the top portion of pump chamber inlet 121, or pressing thin film inlet cover 126, if present, against the top portion of pump chamber inlet 121, and preventing flow between the interior of pump chamber 120 and the interior of flexible pouch 102. If chamber inlet closure 127 is not present, and only thin film inlet cover 126 is present, the downward stroke of flexible actuator 123 causes thin inlet cover 126 to press against the top portion of pump chamber inlet 121, preventing flow between the interior of pump chamber 120 and the interior of flexible pouch 102.

As mentioned briefly above, fluid exit channel 130 is in fluid communication with pump chamber 120. Fluid exit channel 130 includes an intermediate chamber 131 and an outlet channel 132. Intermediate chamber 131 opens at one end into pump chamber 120, and at the opposite end to outlet channel 132. The opposite end of outlet channel 132 comprises fluid outlet 133 from which fluid exits fluid dispenser 100. A flange is positioned around the base of circumferential wall 125 of flexible actuator 123, and a portion of the flange is extended in flexible flange extension 128. Flexible flange extension 128 is positioned over intermediate chamber 131 and, in the at-rest position shown in FIG. 24, lies flat on intermediate chamber 131, acting as a valve to selectively prevent flow of fluid between outlet channel 132 and pump chamber 120. The bottom surface of flexible flange extension 128 is configured to mate with the top surface of the corresponding surface of the bottom piece 112. According to one embodiment, both surfaces are flat. According to other embodiments, each surface may have a profile that is complementary to the other so that they form a seal when they are pressed together. Likewise, the end of intermediate chamber 131 that meets outlet channel 132 preferably opens into an acute bend 134, such that fluid that is being dispensed must travel through a non-straight path as it travels through fluid dispenser 102.

A movable cap 140 may be provided at the end of fluid exit channel 130 and is preferably hinged thereto, such that movable cap 140 may be selectively positioned over fluid outlet 133 to close the same, or may be pivoted to the open position shown in FIG. 24 when the fluid dispenser 100 is in use. Movable cap 140 may be molded integrally with bottom piece 112 or it may be manufactured separately and affixed to a portion of bottom piece 112.

FIG. 25 shows a variety of views of the exterior structure of dispensing assembly 110 as described above.

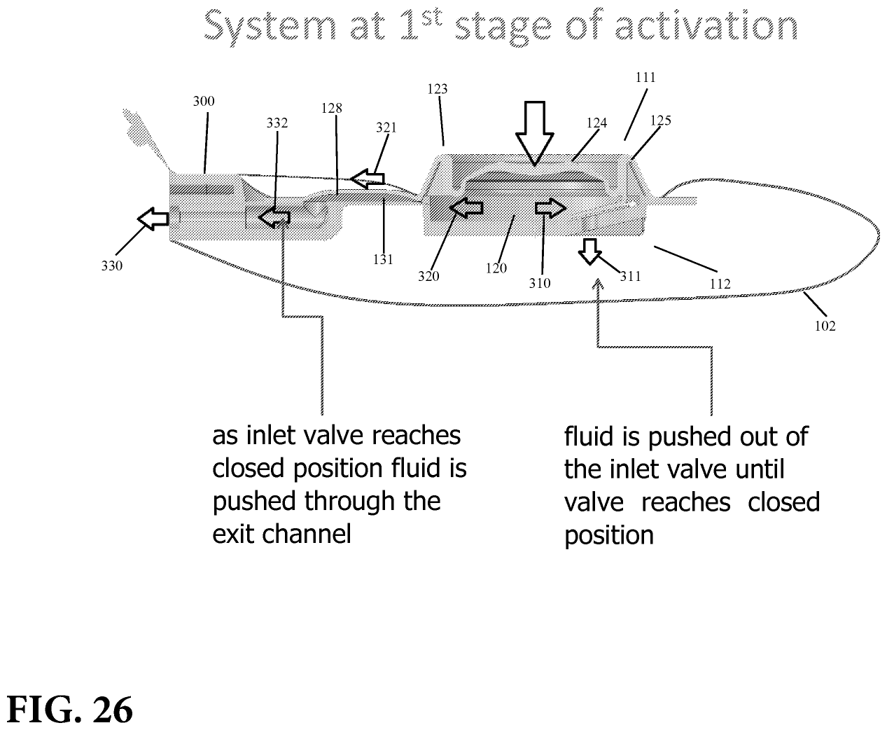

Next, and with regard to FIGS. 26-30, fluid dispenser 100 is shown in various stages of a dispensing operation. First, FIG. 26 shows fluid dispenser 100 at a first stage of activation, in which the dome 124 of flexible actuator 123 is being depressed downward and into pump chamber 120. As bulb 124 starts its downward stroke, fluid that is positioned within pump chamber 120 flows in two directions from pump chamber 120. Namely, as the pressure in pump chamber 120 is not yet sufficient to have closed thin film inlet cover 126 or to have moved pump chamber inlet closure 127, fluid flows in the direction of arrows 310 and 311 from pump chamber 120 into the interior of flexible pouch 102. Simultaneously, fluid flows from pump chamber 120 in the direction of arrows 320-322 into intermediate chamber 131, lifting flexible flange extension 128 to the raised position shown in FIG. 26 so as to allow fluid to flow through intermediate chamber 131, and ultimately to outlet channel 132 where it exits fluid dispenser 100 (arrow 330).

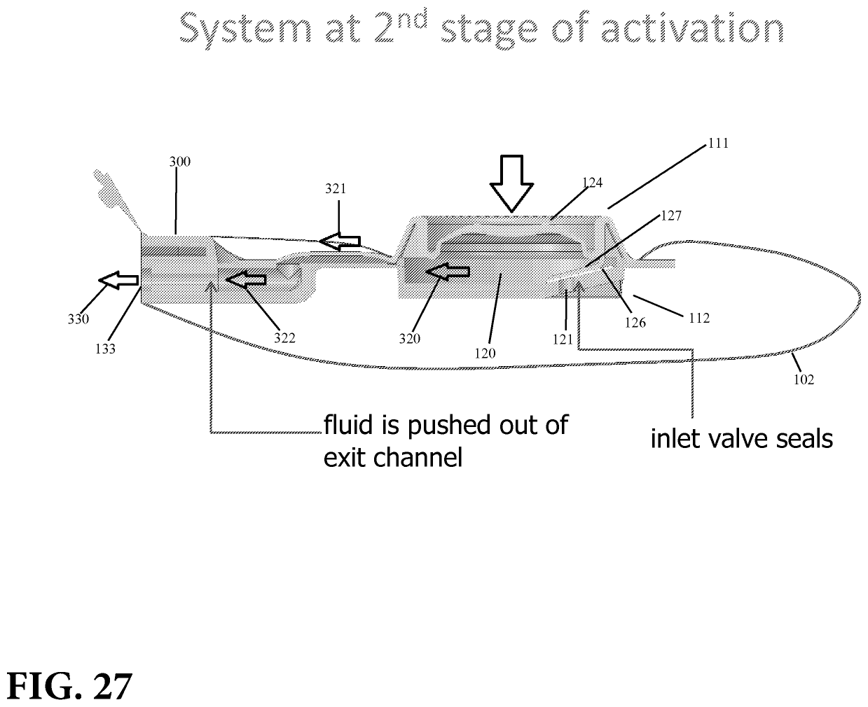

Next, as shown in FIG. 27, as bulb 124 is pressed further downward into pump chamber 120, and as the pressure within pump chamber 120 thus increases, pump chamber inlet closure 127 is directed downward toward pump chamber inlet 121 and, along with pressing thin film inlet cover 126 against pump chamber inlet 121, closes off pump chamber inlet 121 to prevent further fluid flow between the interior of pump chamber 120 and the interior of flexible pouch 102. In this condition, fluid flows only in the direction of arrows 320-322 through fluid exit channel 130 and outward through fluid outlet 133 in the direction of arrow 330.

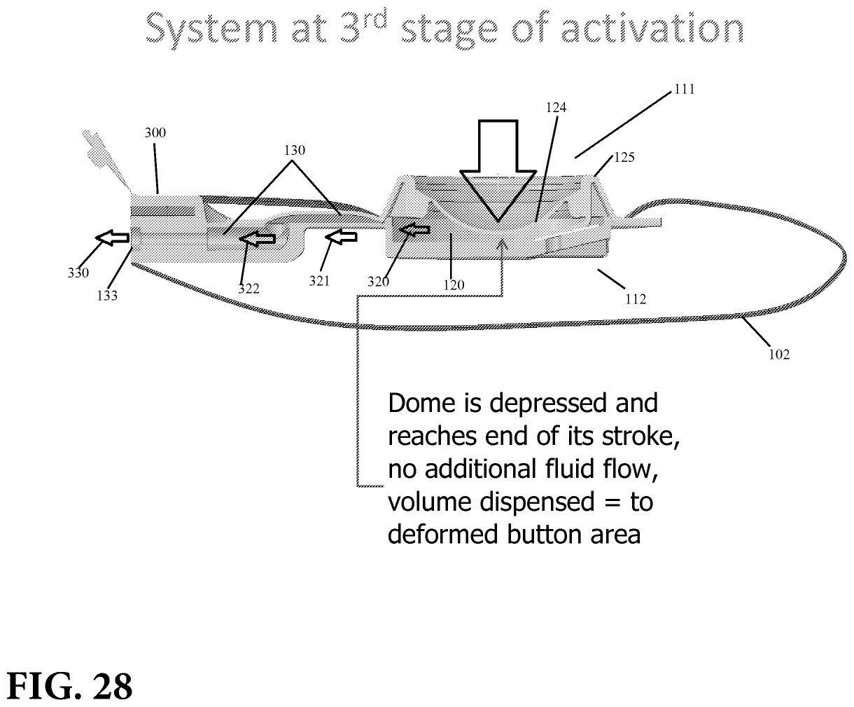

Next, and as shown in FIG. 28, as bulb 124 is pressed further down into pump chamber 120, it reaches its maximum stroke and completes the dispensing of a portion of the volume of the fluid that was originally within pump chamber 120, again causing fluid to flow only in the direction of arrows 320-322 through fluid exit channel 130 and outward through fluid outlet 133 in the direction of arrow 330. Notably, the volume of fluid that is so dispensed from fluid dispenser 100 is thus the volume that has been displaced by the downward stroke of bulb 124, less the volume that returns into flexible pouch 102 through pump chamber inlet 121 (as shown in FIG. 26). Thus, the volume of fluid dispensed is less than the fluid present in the pump chamber. A portion of the fluid that was originally present in pump chamber 120 at the start of the dispensing operation (i.e., that volume of fluid present in the dispensing assembly 110 in FIG. 24) thus remains in pump chamber 120 at the completion of the downstroke of bulb 124, such that pump chamber 120 remains partially primed for the next desired dispensing operation. Likewise, by maintaining some amount of fluid in pump chamber 120, the amount of fluid that is sucked back into pump chamber 120 from fluid exit channel 130 is likewise minimized, in turn minimizing air pockets in the fluid that is dispensed in the next dispensing operation.

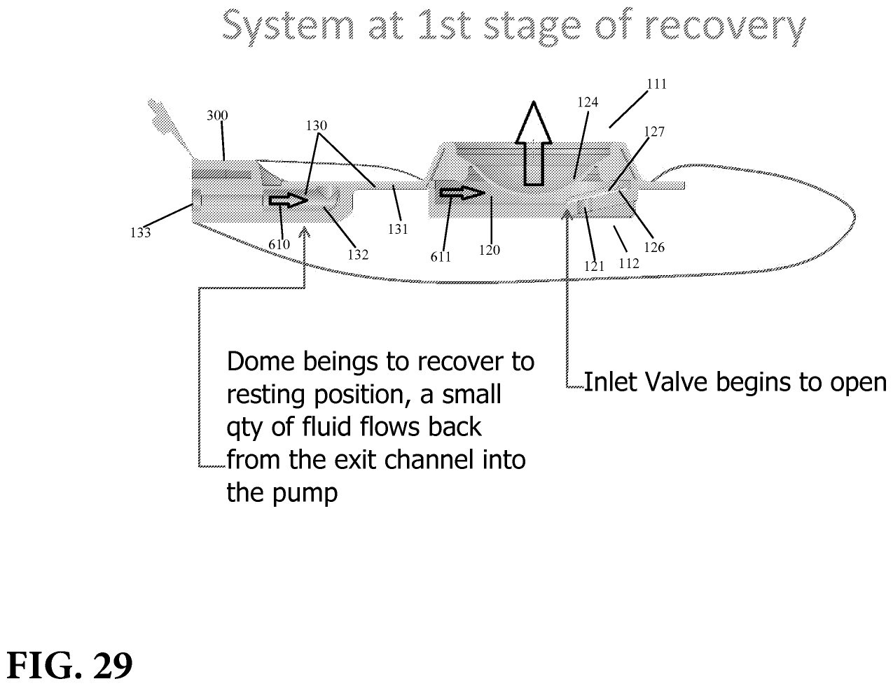

Next, and with reference to FIG. 29, after fluid has been dispensed, dispensing assembly 110 is configured to proceed through a recovery process to return it to the condition reflected in FIG. 24. Namely, bulb 124 of flexible actuator 123 is allowed to rise back to its at-rest position that is reflected in FIG. 24. As bulb 124 begins to rise, a small amount of fluid in fluid exit channel 130 flows back toward pump chamber 120 in the direction of arrows 610 and 611.

During this process, flexible flange extension 128 of flexible actuator 123 is preferably in its at-rest position (collapsed against intermediate chamber 131), thus limiting the flow of fluid from outlet channel 132 back toward pump chamber 120. Likewise during this process, pump chamber inlet closure 127 begins to lift off of pump chamber inlet 121, in turn allowing thin film inlet cover 126 to lift off of pump chamber inlet 121 so as to allow fluid to begin flowing into the interior of pump chamber 120 from the interior of flexible pouch 102.

According to a particular and unexpected advantage of the embodiments, due to the fact that fluid passage 131 and flexible flange extension 128 are situated inside of the flexible pouch, large amounts of pressure may be applied to flexible pouch 102 without causing the unintended escape of fluid from the dispensing assembly because any pressure applied to flexible pouch 102 will translate to the outside of flexible flange extension 128, sealing it against bottom portion of fluid passage 131 and preventing the movement of fluid. Indeed, the more pressure that is applied to flexible pouch 102, the more tightly that flexible flange extension 128 is pressed against the bottom of fluid passage 131. This represents a significant advantage over prior art dispensing devices in which a sufficient amount of pressure applied, intentionally or unintentionally, to the flexible pouch would cause one or more valves to reverse or otherwise fail, causing liquid to unintentionally exit the dispensing device, potentially resulting in the ruin or soiling of neighboring fabrics and other materials.

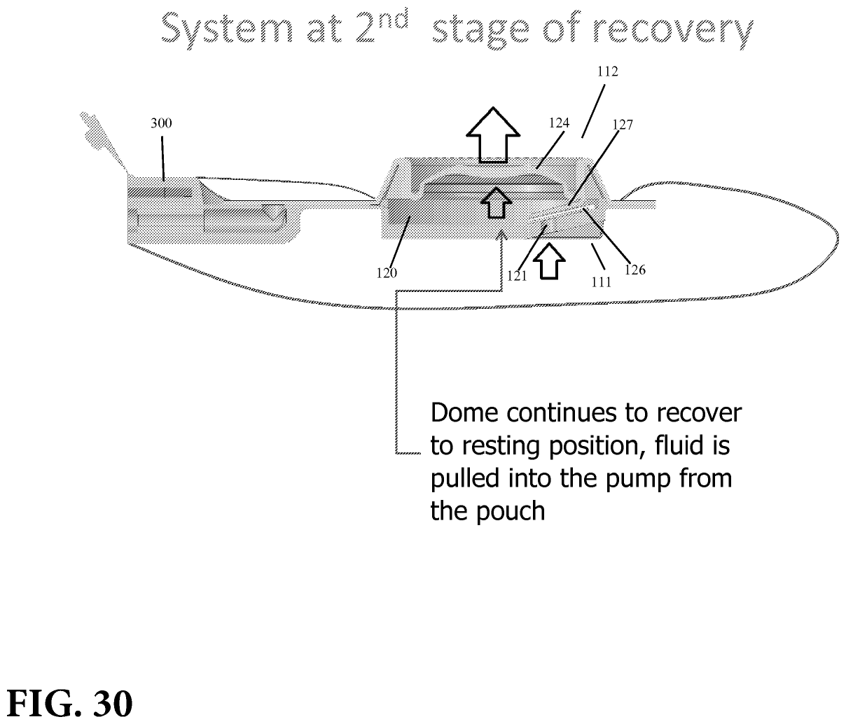

Next, and as shown in FIG. 30, bulb 124 continues to rise towards its at-rest position (shown in FIG. 24), and as it rises preferably pulls additional fluid into pump chamber 120 only through pump chamber inlet 121. Specifically, as bulb 124 rises, it further raises pump chamber inlet closure 127 away from the top of pump chamber inlet 121, allowing fluid to flow from the interior of flexible pouch 102 through pump chamber inlet 121, past thin film inlet cover 126 and into pump chamber 120 until bulb 124 reaches the end of its recovery stroke (i.e., the at-rest position reflected in FIG. 24). At this stage, fluid dispensing 100 is ready for the next dispensing operation the next time that a user engages flexible actuator 123.

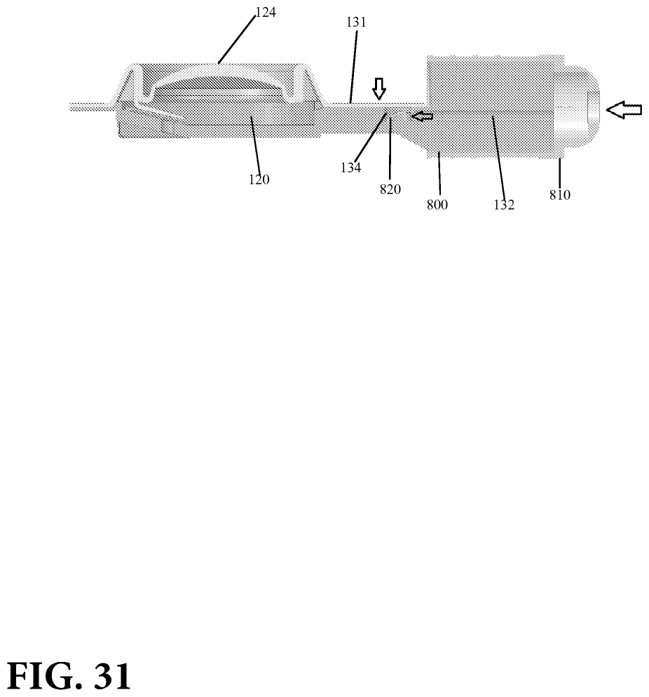

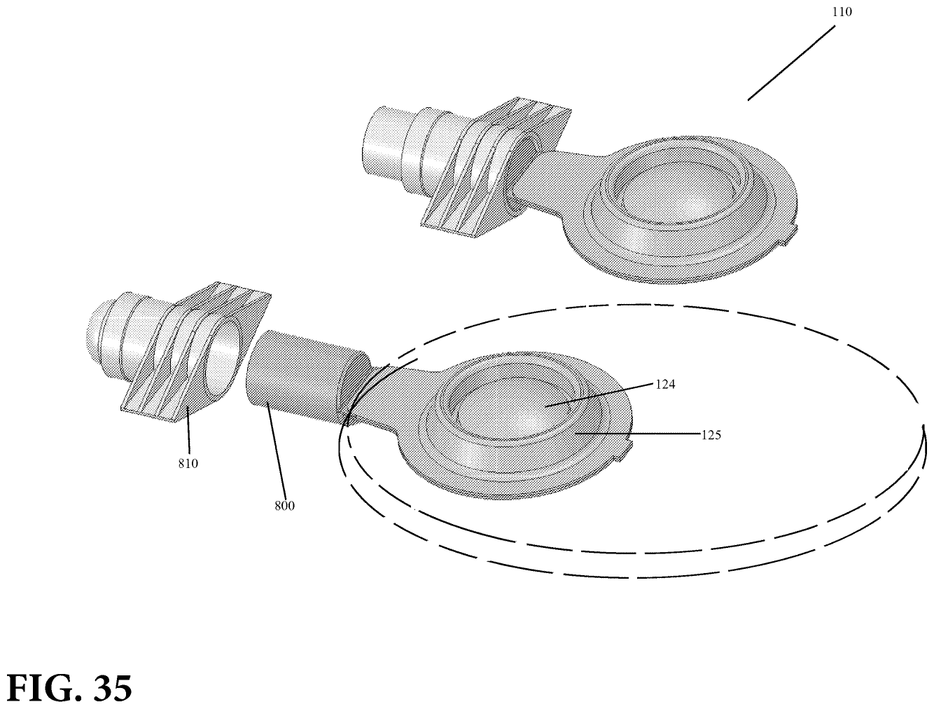

With regard to further aspects of an embodiment, and with reference to FIGS. 31-35, the exterior of fluid exit channel 130 may be provided a generally cylindrical head 800 configured for insertion into a cylindrical cavity within a mating part 810. Mating part 810 may be configured for mating with any of a variety of other devices, such as (by way of non-limiting example) a container having a fluid or other substance therein intended for mixing with the contents of the flexible pouch 102 (not shown in FIGS. 31-34, and shown in phantom in FIG. 35) to which the dispensing assembly 110 is attached.

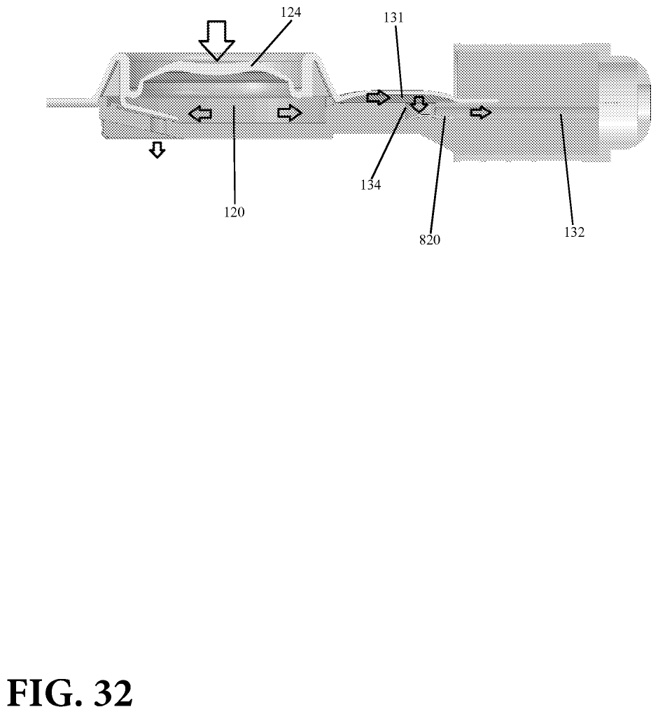

As shown in FIG. 31, in order to further restrict the possible flow of fluid from outlet channel 132 into intermediate channel 131 (and thus back into pump chamber 120), which backflow could in this case possibly include fluid or other material from a container with which the contents of flexible pouch 102 are to be mixed, a flexible flap valve 820 may be provided in the acute bend portion 134 of outlet channel 132. Flexible flap valve 820 is configured so as to bend downward, as shown in FIG. 32, so as to allow fluid to flow from pump chamber 120, through intermediate chamber 131, and into outlet channel 132 for dispensing when bulb 124 is pushed downward. However, when bulb 124 is returning to its at-rest position, flexible flap valve 820 is pulled upward to the position shown in FIG. 31 so as to block fluid from flowing back into intermediate chamber 131.

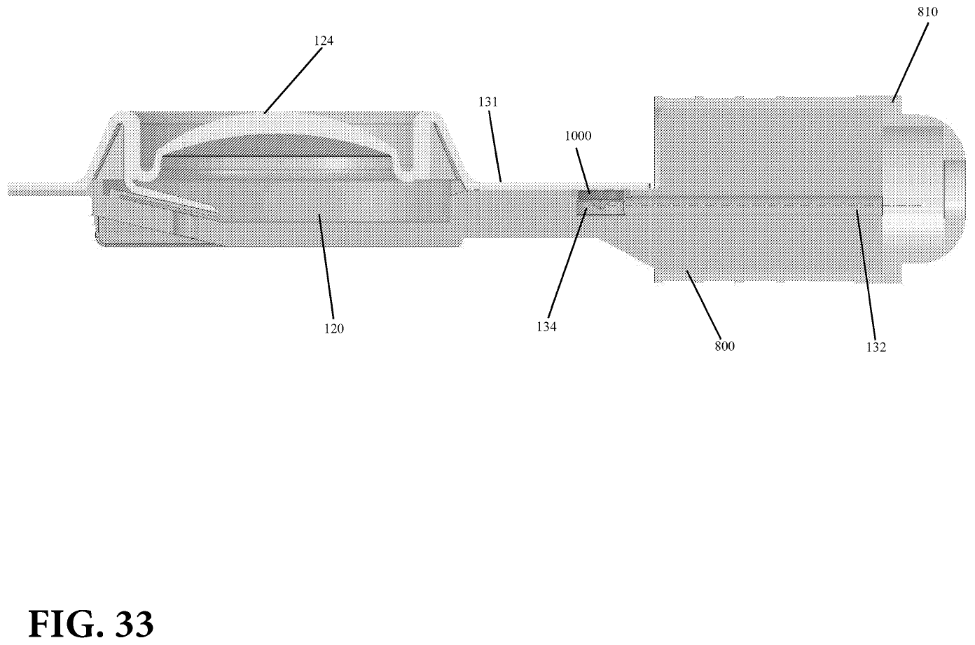

Similarly, and as shown in FIG. 33, instead of providing a flexible flap valve, a duck bill valve 1000 may be positioned at the top of acute bend portion 134 of outlet channel 132. Such a duck bill valve 1000 will likewise serve as a one-way valve, allowing fluid to flow from pump chamber 120 and intermediate chamber 131 into outlet channel 132, while preventing fluid from flowing in the opposite direction

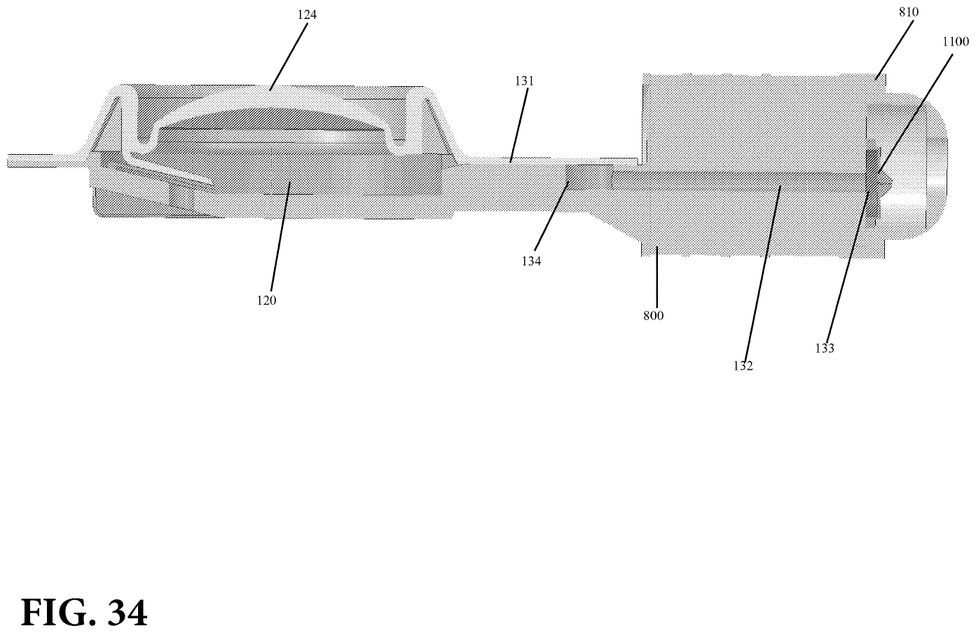

As still another alternative to prevent back flow of fluid from a container with which the contents of flexible pouch 102 are to be mixed, and as shown in FIG. 34, a duck bill valve 1100 may be positioned at the fluid outlet 133 of outlet channel 132, thus preventing any mixing of external fluids with the fluid contained in flexible pouch 102 anywhere within dispensing assembly 110.

While valves 820, 1000, and 1100 are described herein as being incorporated in the configurations that employ cylindrical head 800, it is noted that such valves may likewise be incorporated into the assemblies shown in FIGS. 24-30 to provide additional flow control without departing from the spirit and scope of the disclosure.

According to alternative “floating” embodiments, shown in FIGS. 35-40, the dispensing assembly may be situated entirely inside the flexible pouch. The floating embodiments, described below, can be manufactured on existing machinery, are simpler and therefore less expensive to manufacture, and have no pump/dispensing assembly protruding externally from the product.

FIG. 36 shows a fluid dispenser 100 according to one alternative embodiment in which the dispensing assembly 110 is located inside the flexible pouch 102. According to one alternative embodiment, the dispensing assembly itself is not connected to any portion of the flexible pouch 102. According to this embodiment, the dispensing assembly 110 is only indirectly connected to the flexible pouch, through mating part 810. This embodiment may be manufactured by snapping the head 800 of the dispensing assembly into the throat 811 of the mating part 810, sandwiching the mating part 810 and dispensing assembly 110 between upper and lower films 102′, 102″, and sealing the upper and lower films to one-another along a perimeter except at the mating part 810, where the upper and lower films 102′, 102″ are adhered or otherwise affixed in a leak-proof manner to opposite sides of the mating part 810. According to another manufacturing embodiment, upper and lower films 102′, 102″ may first be adhered or sealed to the mating part 810 and to one-another along a majority of a perimeter; the dispensing assembly 110 may be inserted to the interior of the pouch 102 formed by the mating of the upper and lower films 102′, 102″ through an unsealed section of the pouch 102 and mated with the mating part 810, and the unsealed section may then be closed by adhesive, heat sealing or other method.

According to either manufacture method described above, or according to any other method, the head 800 of dispensing assembly may be configured with a series of ribs and/or detents 801. According to a further alternative embodiment, the interior surface of the mating part throat 811 may be configured to have complementary or matching detents, ribs, or other features to receive the neck of the dispensing assembly in a tight fitting and/or quantum step fashion.

According to some internal pump embodiments, the dispensing assembly may be configured the same or substantially the same as the dispensing assemblies described herein with respect to FIGS. 24-35.

According to other internal pump embodiments, the dispensing assembly may be configured substantially the same as the dispensing assemblies described herein with respect to FIGS. 24-25, except that where FIGS. 24-25 show both a thin cover 126 and pump chamber inlet closure 127 positioned over pump chamber inlet 121, the inlet valve is a membrane only structure.

According to additional aspects of the embodiments illustrated in FIGS. 24-40, a fluid dispending device is provided, including a flexible actuator comprising a flexible dome, a flange extension, and a raise circumferential wall surrounding said flexible dome and connected to said flexible dome at a lower end of the raised circumferential wall; a base portion comprising a bottom portion of a pump chamber inlet; said flexible actuator mated to said base portion so that said flexible dome and said bottom portion of the pump chamber form a pump chamber, said pump changer having an outlet; a dispensing head comprising a fluid outlet channel in fluid communication with said outlet; an inlet valve positioned in proximity to said pump chamber inlet to selectively permit fluid flow through the pump chamber inlet, said inlet valve comprising an inlet closure attached at one end of the lower end of the raised circumferential wall of the flexible actuator allowing a remaining portion of said closure to move between open and closed positions over the pump chamber inlet; and wherein said inlet valve comprises a flap integrally formed with said flexible actuator and a flexible film attached to said bottom portion.

According to additional aspects of the embodiments illustrated in FIGS. 24-40, a fluid dispensing device is provided, including a flexible actuator comprising a flexible dome, a flange extension, and a raised circumferential wall surrounding said flexible dome and connected to said flexible dome at a lower end of the raised circumferential wall; a base portion comprising a bottom portion of a pump chamber and a pump chamber inlet; said flexible actuator mated to said base portion so that said flexible dome and said bottom portion of the pump chamber form a pump chamber, said pump chamber having an outlet; a dispensing head comprising a fluid outlet communication with said outlet; an inlet valve position in proximity to said pump chamber inlet to selectively permit fluid flow through the pump chamber inlet, said inlet valve comprising an inlet closure attached at one end to the lower end of the raised circumferential wall of the flexible actuator allowing a remaining portion of said closure to move between open and closed positions over the pump chamber inlet; and a flexible fluid pouch having two opening, wherein a first opening is sealed around said flexible actuator, and a second opening is sealed around said dispensing head, and wherein said flexible flange extension and all of said bottom portion reside in an interior of said flexible fluid pouch.

According to additional aspects of the embodiments illustrated in FIGS. 24-40, a fluid dispensing device is configured so that increases in pressure on said flexible fluid pouch cause said outlet to seal more tightly against unintentional release of fluid.

According to additional aspects of the embodiments illustrated in FIGS. 24-40, a fluid dispensing device is provided, including a flexible actuator comprising a flexible dome, a flange extension, and a raised circumferential wall surrounding said flexible dome and connected to said flexible dome at a lower end of the raised circumferential wall; a base portion comprising a bottom portion of a pump chamber and a pump chamber inlet; said flexible actuator mated to said base portion so that said flexible dome and said bottom portion of the pump chamber form a pump chamber, said pump chamber having an outlet; a dispensing head comprising a fluid outlet communication with said outlet; an inlet valve position in proximity to said pump chamber inlet to selectively permit fluid flow through the pump chamber inlet, said inlet valve comprising an inlet closure attached at one end to the lower end of the raised circumferential wall of the flexible actuator allowing a remaining portion of said closure to move between open and closed positions over the pump chamber inlet; and a flexible fluid pouch having a single opening, said opening sealed around said dispensing head, wherein the entirety of said flexible actuator and said bottom portion reside in the interior of said flexible fluid pouch.

According to additional aspects of the embodiments illustrated in FIGS. 24-40, the fluid dispensing device according is configured to dispense a volume of fluid that is less than the volume of said pump chamber.

According to additional aspects of the embodiments illustrated in FIGS. 24-40, said pump chamber outlet permits the flow of fluid toward the fluid outlet channel during activation of the flexible dome, and toward the pump chamber when the flexible dome is released.

According to additional aspects of the embodiments illustrated in FIGS. 24-40, said inlet valve initially permits fluid flow away from the pump chamber during activation of the flexible dome, and permits the flow of fluid into the pump chamber following release of the flexible dome.

According to additional aspects of the embodiments illustrated in FIGS. 24-40, said inlet valve is attached to said base portion.

According to additional aspects of the embodiments illustrated in FIGS. 24-40, said device defining a flow path having an acute angle between said outlet and said fluid outlet channel.

According to additional aspects of the embodiments illustrated in FIGS. 24-40, a fluid dispensing device is disclosed having a flexible fluid pouch and a dispensing assembly, the dispensing assembly having a top piece and a bottom piece, the top piece having a flexible actuator including a flexible dome and a flexible flange extension, the bottom piece having a bottom portion of a pump chamber, a pump chamber inlet, a chamber inlet valve, and a bottom portion of an outlet valve, the top piece and the bottom piece mated together to form a pump chamber and an outlet valve, the device configured to allow bidirectional fluid flow through either or both inlet and outlet valves, and to dispense a volume of fluid that is less than the volume of the pump chamber. The flexible pouch may have a single opening or two openings. In the case of a single opening, the entire dispensing assembly is in the interior of the pouch. In the case of two openings, only the flexible dome extends to the exterior of the pouch. The outlet valve is configured so that the more pressure that is applied to the flexible fluid pouch, the more tightly the outlet valve seals.

While the present invention has been particularly described, in conjunction with one or more specific embodiments, it is evident that many alternatives, modifications and variations will be apparent to those skilled in the art in light of the foregoing description. It is therefore contemplated that the appended claims will embrace any such alternatives, modifications and variations as falling within the true scope and spirit of the present invention.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

Those of skill in the art will appreciate that various example embodiments are shown and described herein, each having certain features in the particular embodiments, but the present disclosure is not thus limited. Rather, the present disclosure can be modified to incorporate any number of variations, alterations, substitutions, combinations, sub-combinations, or equivalent arrangements not heretofore described, but which are commensurate with the scope of the present disclosure. Additionally, while various embodiments of the present disclosure have been described, it is to be understood that aspects of the present disclosure may include only some of the described embodiments. Accordingly, the present disclosure is not to be seen as limited by the foregoing description, but is only limited by the scope of the appended claims.

1. An apparatus for dispensing fluid, comprising:
a flexible and collapsible fluid container;
a fluid dispenser pump connected to the flexible and collapsible fluid container configured to draw fluid from the flexible and collapsible fluid container into the fluid dispenser pump and to dispense fluid through a fluid delivery channel to an exit port upon actuation of the pump; and
a sensor located in the apparatus,
wherein:
(i) the dispenser pump has an actuator that comprises a dose delivery button, and the sensor is configured to actuate the dispenser pump upon physical contact of the dose delivery button by a user; or
(ii) the sensor is configured to actuate the dispenser pump upon the first voice command received by the sensor by a user, and the sensor is further configured to adjust an amount of a dose of fluid to be dispensed upon the sensor receiving the second voice command by the user; or
(iii) upon the sensor receiving the biometric identification information, the sensor is configured to unlock; actuation of the dispenser pump; and adjustment of the amount of the dose of fluid to be dispensed by the fluid dispenser pump.

2. A method of dispensing fluid dispensed from the apparatus of claim 17, comprising:
contacting, by the user, the dose delivery button;
signaling the sensor to actuate the fluid dispensing pump; and
dispensing the fluid from the flexible and collapsible fluid container through the exit port.

3. A fluid dispensing apparatus comprising:
a base unit having a flat and continuous bottom surface, a top surface having a raised circumferential wall, and a center area;
a flexible dosing dome configured to sit inside the center area of the base unit to form a pump chamber;
the bottom surface of the base unit having a fluid inlet opening to the pump chamber to permit the flow of fluid there-through, the base also defining a fluid delivery channel between the pump chamber and a fluid delivery outlet opening on an outside surface of the base unit;
a combination dial and button having the shape of a cylinder with a closed top and open bottom, a bottom portion of the combination dial and button configured to sit inside a channel defined by the base unit top surface and flexible dome, button including a graphical user interface (GUI) in communication with a sensor to adjust by a user an amount of a dose of fluid to be dispensed; and
the sensor located in the apparatus and further configured to actuate the pump chamber upon physical contact of the dose delivery button by the user.

4. A method of dispensing and selecting an amount of fluid from the apparatus claim 3, comprising:
selecting from the GUI on the button by the user the adjustment state of the actuator;
signaling to the sensor the adjustment state of the actuator to adjust the amount of the dose of fluid dispensed upon actuation;
contacting, by the user, the dose delivery button to send a signal to the sensor; and
signaling actuation of the pump chamber by the sensor upon contact of the button, wherein the pump chamber dispenses the selected dose of fluid to the outside surface of the base unit.

5. An apparatus for dispensing and monitoring fluid dispensed comprising:
a flexible and collapsible fluid container;
a fluid dispenser pump connected to the flexible and collapsible fluid container configured to draw fluid from the flexible and collapsible fluid container into the fluid dispenser pump and to dispense fluid through a fluid delivery channel to an exit port upon actuation of the pump;
a sensor located in the apparatus; and
a wireless transmission module in communication with the sensor,
wherein:
(i) the sensor is configured to actuate the dispenser pump upon the sensor receiving a first voice command by a user, the sensor is further configured to measure a physical change upon receipt of a second voice command by the user, the physical change comprising information on the amount of fluid dispensed; and the wireless transmission module is configured to communicate a physical change to a remote device upon receipt of second voice command by the user; or
(ii) the dispenser pump has an actuator which, upon actuation by a user, actuates the fluid dispenser pump to dispense a dose of fluid: the sensor is configured to measure the physical change upon the actuation of the actuator by the user, the physical change comprising the information on the amount of fluid dispensed; and the wireless transmission module is configured to communicate the physical change, to a remote device, only upon the actuation of the actuator by the user.

6. A method of dispensing and monitoring fluid dispensed from the apparatus of claim 18, comprising:
signaling by the user, to the sensor, the first voice command or the second voice command,
wherein:
the sensor signals actuation of the fluid dispenser pump to dispense the fluid from the flexible and collapsible fluid container through the exit port in response to the sensor receiving the first voice command; and
the sensor wirelessly transmits information on the amount of fluid dispensed from the sensor to a remote device in response to the sensor receiving the second voice command by the user.

7. The apparatus of claim 1, wherein
the sensor is configured to actuate the dispenser pump upon the first voice command received by the sensor by the user; and
the sensor further is configured to adjust the amount of the dose of fluid to be dispensed upon the sensor receiving the second voice command by the user.

8. A method of dispensing and selecting an amount of fluid dispensed from the apparatus of claim 7, comprising:
signaling by the user, to the sensor, the first voice command or the second voice command,
wherein:
the sensor signals actuation of the fluid dispenser pump to dispense the fluid from the flexible and collapsible fluid container through the exit port in response to the sensor receiving the first voice command by the user; and
the sensor to adjusts the amount of the dose of fluid to be dispensed in response to the sensor receiving second voice command by the user.

9. The apparatus of claim 1, wherein:
upon the sensor receiving the biometric identification information, the sensor is configured to unlock: actuation of the dispenser pump; adjustment of the amount of the dose of fluid to be dispensed by the fluid dispenser pump.

10. A method of unlocking and dispensing fluid dispensed from the apparatus of claim 9, comprising:
detecting biometric identification information by the sensor;
analyzing the biometric identification information to detect an initial biometric identification information; and
in response to detecting the initial biometric identification information, allowing the user of the fluid dispensing apparatus to actuate the fluid dispenser pump to one or more of:
dispense the fluid from the flexible and collapsible fluid container through the exit port; or adjust the amount of the dose of fluid to be dispensed by the fluid dispenser pump.

11. The apparatus of claim 5, wherein
the dispenser pump includes the actuator, which, upon actuation by a user, actuates the fluid dispenser pump to dispense the dose of fluid;
the sensor is configured to measure the physical change upon the actuation of the actuator of the fluid dispenser pump by the user, the physical change comprising the information on the amount of fluid dispensed; and
the wireless transmission module is configured to communicate the physical change, to a remote device, only upon the actuation of the actuator of the fluid dispenser pump by the user.

12. A method of assembling the fluid dispensing apparatus of claim 20, comprising:
securing the flexible cover to an exterior surface of the fluid container such that the flexible cover forms the protective surface around the fluid container.

13. The apparatus of claim 1, further comprising:
a flexible cover secured to an exterior surface of the fluid container,
the cover including sidewalls around a periphery of the flexible cover to encase the fluid container therein,
wherein the sidewalls of the flexible cover include a flat end, such that the fluid container is configured for being placed on an end thereof while on a substantially flat surface.

14. A method of assembling the fluid dispensing apparatus of claim 13, comprising:
securing the flexible cover to an exterior surface of the fluid container such that the flexible cover forms a protective surface around the fluid container.

15. The apparatus of claim 11, wherein:
actuation of the dispensing pump provides the sensor with dose information including one or more of: dose amount; or time of day,
whereby the apparatus is configured to predict the dose information on a future actuation of the dispensing pump transmitted to the remote device by the wireless transmission module.

16. A method of dispensing fluid dispensed from the apparatus of claim 15, comprising:
actuating, by the user, the fluid dispenser pump to dispense the fluid from the flexible and collapsible fluid container through the exit port;
upon actuation of the fluid dispenser pump, sensing by the sensor, information on the amount of fluid dispensed from the exit port;
storing the information on the amount of fluid dispensed from the exit port;
determining suggested dose information in response to the information on the amount of fluid dispensed from the exit port stored by the sensor; and
providing the suggested dose information to the remote device by the wireless transmission module.

17. The apparatus of claim 1, wherein:
the dispenser pump has the actuator that comprises the dose delivery button; and
the sensor is configured to actuate the dispenser pump upon physical contact of the dose delivery button by the user.

18. The apparatus of claim 5, wherein:
the sensor is configured to actuate the dispenser pump upon the first voice command received by the sensor by the user;
the sensor is further configured to measure the physical change upon the sensor receiving the second voice command by the user, the physical change comprising information on the amount of fluid dispensed; and
the wireless transmission module is configured to communicate the physical change to the remote device upon the sensor receiving the second voice command by the user.

19. The apparatus of claim 11, wherein:
the actuator is a dose delivery button.

20. The apparatus of claim 19, comprising:
a flexible cover secured to an exterior surface of the fluid container to form a protective surface around the fluid container.
